(19) United States
(12) Patent Application Publication
(10) Pub. No.: US 2014/0221286 A1
(43) Pub. Date: Aug. 7, 2014

(54) SODIUM CHANNEL BLOCKERS REDUCE GLUCAGON SECRETION

(71) Applicant: Gilead Sciences, Inc., Foster City, CA (US)

(72) Inventors: Luiz Belardinelli, Palo Alto, CA (US); Arvinder Dhalla, Mountain View, CA (US)

(21) Appl. No.: 14/345,893

(22) PCT Filed: Sep. 20, 2012
(86) PCT No.: PCT/US2012/056419

§ 371 Date: Mar. 19, 2014

Related U.S. Application Data
(60) Provisional application No. 61/537,411, filed on Sep. 21, 2011.

(51) Int. Cl. A61K 38/28; A61K 31/138; A61K 31/4458; A61K 31/4965; A61K 31/519; A61K 31/341; A61K 31/49; A61K 31/166; A61K 31/4402; A61K 31/4166; A61K 31/4453; A61K 31/5415; A61K 45/06; A61K 31/64; A61K 31/4015; A61K 31/451; A61K 31/192; A61K 31/4439; A61K 31/167

(52) U.S. Cl. CPC A61K 38/28; A61K 31/167; A61K 31/138; A61K 31/4458; A61K 31/4965; A61K 31/519; A61K 31/341; A61K 31/49; A61K 31/166; A61K 31/4402; A61K 31/4166; A61K 31/4453; A61K 31/5415; A61K 45/06; A61K 31/64; A61K 31/4015; A61K 31/451; A61K 31/192; A61K 31/4439
USPC 514/6.5; 514/626; 514/651; 514/331; 514/255.06; 514/249; 514/471; 514/305; 514/619; 514/357; 514/391; 514/225.2; 514/592; 514/423; 514/563; 514/342; 564/194; 564/354; 546/233; 546/333; 544/407; 544/260; 548/321.1; 435/375

(57) ABSTRACT

It is discovered that sodium-channel blockers inhibit the secretion of glucagon from pancreatic alpha cells. The present disclosure, based on such discoveries, provides compositions and methods for the treatment of hyperglycemia and related diseases and conditions with Na-channel blockers.

### CROSS REFERENCE TO RELATED APPLICATIONS

This application claims the benefit under 35 U.S.C. §119(e) of U.S. Provisional Application Ser. No. 61/537,411 filed Sep. 21, 2011, the content of which is incorporated by reference in its entirety into the present disclosure.

### FIELD

Methods are provided for treating diabetes, lowering plasma levels of glucose and HbA1c and delaying onset of diabetic complications in a diabetic or pre-diabetic patient.

### BACKGROUND

Diabetes mellitus is a disease characterized by hyperglycemia; altered metabolism of lipids, carbohydrates and proteins; and an increased risk of complications from vascular disease. Diabetes is an increasing public health problem, as it is associated with both increasing age and obesity.

There are two major types of diabetes mellitus: 1) Type I, also known as insulin dependent diabetes (T1DM), and 2) Type II, also known as insulin independent or non-insulin dependent diabetes (T2DM or NIDDM). T1DM is due to insufficient amounts of circulating insulin whereas type 2 diabetes is due to a decrease in the response of peripheral tissue to insulin. Ultimately, insulin deficiency is present in both types of diabetes.

T1DM results from the body's failure to produce insulin, the hormone that “unlocks” the cells of the body, allowing glucose to enter and fuel them. The complications of TIDM include heart disease and stroke; retinopathy (eye disease); kidney disease (nephropathy); neuropathy (nerve damage); as well as maintenance of good skin, foot and oral health.

T2DM results from the body's inability to either produce enough insulin or the cell's inability to use the insulin that is naturally produced by the body. The condition where the body is not able to optimally use insulin is called insulin resistance. In patients with T2DM, stress, infection, and medications (such as corticosteroids) can also lead to severely elevated blood sugar levels. Accompanied by dehydration, severe blood sugar elevation in patients with T2DM can lead to an increase in blood osmolality (hyperosmolar state). This condition can lead to coma.

Insulin lowers the concentration of glucose in the blood by stimulating the uptake and metabolism of glucose by muscle and adipose tissue. Insulin stimulates the storage of glucose in the liver as glycogen, and in adipose tissue as triglycerides. Insulin also promotes the utilization of glucose in muscle for energy. Thus, insufficient insulin levels in the blood, or decreased sensitivity to insulin, gives rise to excessively high levels of glucose in the blood.

The toxic effects of excess plasma levels of glucose include the glycosylation of other proteins. Glycosylated products accumulate in tissues and may eventually form cross-linked proteins, which cross-linked proteins are termed advanced glycosylation end products. It is possible that non-enzymatic glycosylation is directly responsible for expansion of the vascular matrix and vascular complications of diabetes. For example, glycosylation of collagen results in excessive cross-linking, resulting in atherosclerotic vessels. Also, the uptake of glycosylated proteins by macrophages stimulates the secretion of pro-inflammatory cytokines by these cells. The cytokines activate or induce degradative and proliferative cascades in mesenchymal and endothelial cells respectively.

The glycation of hemoglobin provides a convenient method to determine an integrated and long-term index of the glycemic state. The level of glycosylated proteins reflects the level of glucose over a period of time and is the basis of an assay referred to as the hemoglobin A1c (HbA1c) assay.

Thus, controlling blood glucose levels is a desirable therapeutic goal. A number of oral antihyperglycemic agents are known. Medications that increase the insulin output by the pancreas include sulfonylureas (including chlorpropamide (Orinase®), tolbutamide (Tolinase®), glyburide (Micronase®), glipizide (Glucotrol®), and glimepiride (Amaryl®)) and meglitinides (including reparglinide (Prandin®) and nateglinide (Starlix®)). Medications that decrease the amount of glucose produced by the liver include biguanides (including metformin (Glucophage®). Medications that increase the sensitivity of cells to insulin include thazolidinediones (including troglitazone (Resulin®), pioglitazone (Actos®) and rosiglitazone (Avandia®)). Medications that decrease the absorption of carbohydrates from the intestine include alpha glucosidase inhibitors (including acarbose (Precose®) and miglitol (Glyset®)). Actos® and Avandia® can change the cholesterol patterns in diabetics. Precose® works on the intestine; its effects are additive to diabetic medications that work at other sites, such as sulfonylureas. ACE inhibitors can be used to control high blood pressure, treat heart failure, and prevent kidney damage in people with hypertension or diabetes. ACE inhibitors or combination products of an ACE inhibitor and a diuretic, such as hydrochlorothazide, are marketed. However, a need still remains for more effective, safer treatments.

### SUMMARY

It has been discovered that α-cells of certain diabetic mice have increased glucagon content, express larger Na⁺ current and have increased action potential duration, amplitude and firing frequency as compared to cells from normal mice. These conditions sensitize the cells for increased glucagon secretion. This data suggests that inhibition of abnormal glucagon secretion from α-cells can provide a novel and first-in-class mechanism for the treatment of hyperglycemia and related diseases and conditions, such as diabetes.

The present disclosure further provides data evidencing that various sodium (Na)-channel blockers inhibited the secretion of glucagon from pancreatic islets. Along with the above discovery, the present disclosure provides evidence that sodium-channel blockers can be used to treat hyperglycemia and related diseases and conditions.

In one embodiment, the present disclosure provides a method of reducing the secretion of glucagon from a pancreatic alpha cell, comprising contacting the alpha cell with an agent that suppresses the influx of sodium ions through sodium channels.

In another embodiment the present disclosure provides a method of reducing secretion of glucagon from a pancreatic alpha cell wherein the alpha cell secretes a higher level of glucagon as compared to a normal pancreatic alpha cell.

In another embodiment, the present disclosure provides a method of lowering the plasma level of HbA1c or glucose, delaying onset of diabetic complications, or treating diabetes in a patient, comprising administering to the patient an effective amount of an agent that suppresses the conduction of sodium ions through sodium channels wherein said agent is selected from the group consisting of lidocaine, mexiletine, flecamide, amiloride, triamterene, benzamil, A-803467, quinidine, procainamide, disopyramide, tocamide, phenyloin, encamide, moricizine, and propafenone, a local anesthetic, a class I antiarrhythmic agent, an anticonvulsant, and combinations thereof.

In another embodiment, the present disclosure provides a method for the manufacture of a medicament for use in lowering the plasma level of HbA1c or glucose, delaying onset of diabetic complications, or treating diabetes in a patient, comprising administering to the patient an effective amount of an agent that suppresses the conduction of sodium ions through sodium channels. In some aspect, the agent is selected from the group consisting of lidocaine, mexiletine, flecamide, amiloride, triamterene, benzamil, A-803467, quinidine, procainamide, disopyramide, tocamide, phenyloin, encamide, moricizine, and propafenone, a local anesthetic, a class I antiarrhythmic agent, an anticonsulsant, and combinations thereof.

In another embodiment, the present disclosure provides a method of treating diabetes in a patient, comprising administering to the subject (a) a synergistically therapeutically effective amount of insulin or a drug that increases the production of insulin or sensitivity to insulin and (b) a synergistically therapeutically effective amount of an agent that suppresses the conduction of sodium ions through sodium channels.

Methods of manufacture of medicaments are also provided for implementing various methods in the present disclosure.

### BRIEF DESCRIPTION OF THE FIGURES

FIG. 1 shows that sodium-channel blockers, ranolazine (A), compound A (B) and tetrodotoxin (TTX, C) concentration-dependently reduced low glucose-induced glucagon secretion in rat pancreatic islets. Data are presented as mean±SEM from the number of experiments indicated for each graph where each experimental condition was run in triplicates. *p<0.05, **p<0.01, ***p<0.001 by One-way ANOVA followed by Dunnett's Multiple Comparison test.

FIG. 2 shows that sodium-channel blockers, ranolazine (A), and compound A (B) concentration-dependently reduced low glucose-induced glucagon secretion in human pancreatic islets. Data are presented as mean±SEM from the number of experiments indicated for each graph where each experimental condition was run in triplicates. *p<0.05, ***p<0.001 by One-way ANOVA followed by Dunnett's Multiple Comparison test.

FIG. 3 shows that sodium-channel blockers, ranolazine (A), compound A (B) or TTX (C) concentration-dependently reduced veratridine-induced glucagon secretion in rat pancreatic islets. Data are presented as mean±SEM from the number of experiments indicated for each graph where each experimental condition was run in triplicate. *p<0.05, *p<0.01, ***p<0.001 by One-way ANOVA followed by Dunnett's Multiple Comparison test.

FIG. 4 shows that sodium-channel blockers, ranolazine (A), and compound A (B) concentration-dependently reduced veratridine-induced glucagon secretion in human pancreatic islets. Data are presented as mean±SEM from the number of experiments indicated for each graph where each experimental condition was run in triplicate. *p<0.05, **p<0.01, ***p<0.001 by One-way ANOVA followed by Dunnett's Multiple Comparison test.

FIG. 5 shows that sodium-channel blockers, TTX (A), and compound A (B) concentration-dependently reduced veratridine-induced glucagon secretion in α-TC1 clone 9 cells. Data are presented as mean±SEM from the number of experiments indicated for each graph where each experimental condition was run in triplicate. *p<0.05, **p<0.01, ***p<0.001 by One-way ANOVA followed by Dunnett's Multiple Comparison test.

FIG. 6 shows that sodium-channel blockers significantly reduced epinephrine-induced glucagon secretion in rat pancreatic islets. (A) Effect of various concentrations of epinephrine on glucagon secretion. (B) Effect of ranolazine on epinephrine-induced glucagon secretion. Data are presented as mean±SEM from the number of experiments indicated for each graph where each experimental condition was run in triplicate. *p<0.05, **p<0.01, ***p<0.001 by One-way ANOVA followed by Dunnett's Multiple Comparison test.

FIG. 7 shows that sodium channel blockers significantly reduced arginine-induced glucagon secretion in rat pancreatic islets. (A) Effect of L-arginine on glucagon secretion. (B) Effect of 10 μM ranolazine or 1 μM compound A on arginine-induced glucagon secretion. Data are presented as mean±SEM from the number of experiments indicated for each graph where each experimental condition was run in triplicate. *p<0.05, **p<0.01 by One-way ANOVA followed by Dunnett's Multiple Comparison test.

FIG. 8 shows representative electrical recordings in the absence and presence of 10 μM ranolazine in rat isolated pancreatic α-cell.

FIG. 9 shows voltage-clamp protocol along with representative Na⁺ current traces (A) in the absence (in black) and presence of 10 μM ranolazine (gray) in rat isolated pancreatic α-cell. (B) Summary of inhibition of Na current by ranolazine at −70 and -90 mV holding potential from n=6.

FIG. 10 shows fasting plasma glucose (FPG) (A) and HbA1c (B) in streptozotocin (STZ)-induced diabetic mice treated with vehicle or ranolazine (20 mg/kg, per oral (p.o.), twice daily) for 8 weeks. Animals were fasted for 4 hrs before FPG and HbA1c measurement. B stands for Baseline. Data are presented as mean±SEM. *, p<0.05 vs. STZ+vehicle group by Two-way ANOVA.

FIG. 11 shows representative pancreatic islets with H/E staining (A) and fluorescent staining (B) from normal mice, STZ-induced diabetic mice treated with vehicle or ranolazine for 8 weeks. Red stain (shown as dark gray) is for insulin-expressing β-cells (Cysteine)(20×); green stain (shown as light gray) is for glucagon-expressing α-cells (FITC)(20×).

FIG. 12 shows that sodium channel blockers lower glucose levels in Zucker Diabetic Fatty (ZDF) rats, an animal model of type 2 diabetes. HbA1c (A), FPG (B), normal fasting glucose (NFG) (C) and water consumption (D) in ZDF diabetic rats treated with vehicle, ranolazine, compound A and sitagliptin in Purina 5008 diet for 10 weeks. Data are presented as mean±SEM. *, p<0.05, **, p<0.01, ***, p<0.001 vs. vehicle group by Two-way ANOVA.

FIG. 13 shows representative pancreatic islets stained with fluorescent staining from ZDF diabetic rats treated with vehicle, ranolazine, compound A and sitagliptin in Purina 5008 diet for 10 weeks. Red stains (shown as dark gray) for insulin-expressing β-cells (20×); green stains (shown as light gray) for glucagon-expressing α-cells (20×).

FIG. 14 shows quantification of total islet area (A), insulin-expressing β-cells and glucagon-expressing α-cells in islet (B), pancreatic insulin/glucagon ratio (C) in pancreas from ZDF diabetic rats treated with vehicle, ranolazine, compound A and sitagliptin in Purina 5008 diet for 10 weeks. All sections from fluorescent staining were viewed under fluorescent microscope and the stained areas were digitally photographed at a magnification of 20×. The images taken at different magnification were normalized using the standard ruler grade (S1 Finder Graticule, 68040, Electron Microscopy Science, Hatfield, Pa.). Analyses of islet areas and entire section areas were performed using ImageJ software (NIH, MD). Three sections from each of 6 animals per treatment group were analyzed. Data are presented as mean±SEM. *, p<0.05; **, p<0.01; ***, p<0.001 by One-way ANOVA.

FIG. 15 shows gene expression of sodium channel subtypes in rat and human pancreatic islets. The levels of gene expression of sodium channel subtypes in isolated rat (A) and human (B) pancreatic islets were determined by qPCR and normalized by the expression levels of β-actin. Data are presented as mean±SEM from the number of experiments indicated for each graph where each experimental condition was run in duplicate.

FIG. 16 shows correlation between inhibition of the Na_{V}1.3 (A) and Na_{V}1.7 (B) Na⁺ channel isoforms and glucagon secretion (data from Table 3). Voltage-dependent block (VDB) of Na_{V}10.3 and Na_{V}10.7 was determined by whole-cell voltage-clamp recordings of sodium current using a QPatch 16× automated electrophysiological system in HEK 293 cells overexpressing Nav 1.3 and Nav 1.7 sodium channels, respectively. VDB of peak current was measured using an 8 s conditioning prepulse (to -55 mV for Na_{V}1.3 and to −60 mV for Na_{V}10.7) followed by a test pulse (0 mV, 20 ms). Currents are normalized to the peak current recorded in the absence of drug and expressed as percent inhibition. Glucagon secretion was measured in α-TC1 clone 9 cells by an ELISA assay. Glucagon secretion in the cells was induced by the treatment with veratridine at 30 μM for 1 hour in Krebs-Ringer buffer containing 0.1% BSA. Percent inhibition of glucagon secretion by Na channel blockers was calculated from the reduction of veratridine-induced glucagon secretion in the absence of drug.

### DETAILED DESCRIPTION

Prior to describing this disclosure in greater detail, the following terms will first be defined.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise. Thus, for example, reference to “an additional therapeutic agent” includes a plurality of therapeutic agents.

### 1. DEFINITIONS

Unless defined otherwise, all technical and scientific terms used herein have the same meaning as commonly understood by one of ordinary skill in the art to which this disclosure belongs. As used herein the following terms have the following meanings.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure. “Consisting of” shall mean excluding more than trace elements of other ingredients and substantial method steps. Embodiments defined by each of these transition terms are within the scope of this disclosure.

The term “about” when used before a numerical designation, e.g., temperature, time, amount, and concentration, including range, indicates approximations which may vary by (+) or (−) 10%, 5% or 1%.

The term “contacting an alpha cell” as used herein means administering an agent of the present disclosure such that the agent comes in contact with an alpha cell. In one embodiment, the agent is administered to a patient such that alpha cells in the patient are contacted in vivo by the administration of the agent.

The term “treatment” means any administration of a compound by the method of the disclosure by any delivery means to a patient for purposes including: (i) preventing the disease or complication of the disease, that is causing the clinical symptoms not to develop; (ii) inhibiting the disease progression, that is, arresting the development of clinical symptoms; and/or (iii) relieving the disease, that is, causing the regression of clinical symptoms. By way of example only, treating may include lowering plasma levels of glucose and HbA1c and delaying onset of diabetic complications.

The term “therapeutically effective amount” refers to that amount of a compound suitable for practice of the present technology, such as ranolazine, that is sufficient to effect treatment, as defined above, when administered to a patient in need of such treatment. The therapeutically effective amount will vary depending upon the specific activity or delivery route of the agent being used, the severity of the patient's disease state, and the age, physical condition, existence of other disease states, and nutritional status of the patient. Additionally, other medication the patient may be receiving will effect the determination of the therapeutically effective amount of the therapeutic agent to administer.

“Synergistic” means that the therapeutic effect of a drug, such as insulin or one that increases a subject's production of insulin or sensitivity to insulin, when administered in combination with another drug, such as a sodium channel blocker, (or vice-versa) is greater than the predicted additive therapeutic effects of each of them when administered alone.

The term “synergistically therapeutic amount” typically refers to a less than standard therapeutic amount of one or both drugs, meaning that the amount required for the desired effect is lower than when the drug is used alone. A synergistically therapeutic amount also includes when one drug is given at a standard therapeutic dose and another drug is administered in a less than standard therapeutic dose. For example, one drug could be given in a therapeutic dose and the other could be given in a less than standard therapeutic dose to provide a synergistic result. In some embodiments, both drugs can be administered in a standard therapeutic dose and the synergy results in much higher efficacies.

The term “patient” typically refers to a human patient. However, the term encompasses a “mammal” which includes, without limitation, monkeys, rabbits, mice, domestic animals, such as dogs and cats, farm animals, such as cows, horses, or pigs, and laboratory animals.

As used herein, “pharmaceutically acceptable carrier” includes any and all solvents, dispersion media, coatings, antibacterial and antifungal agents, isotonic and absorption delaying agents and the like that are pharmaceutically acceptable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Intravenous administration” is the administration of substances directly into a vein, or “intravenously.” Compared with other routes of administration, the intravenous (IV) route is the fastest way to deliver fluids and medications throughout the body. An infusion pump can allow precise control over the flow rate and total amount delivered, but in cases where a change in the flow rate would not have serious consequences, or if pumps are not available, the drip is often left to flow simply by placing the bag above the level of the patient and using the clamp to regulate the rate. Alternatively, a rapid infuser can be used if the patient requires a high flow rate and the IV access device is of a large enough diameter to accommodate it. This is either an inflatable cuff placed around the fluid bag to force the fluid into the patient or a similar electrical device that may also heat the fluid being infused. When a patient requires medications only at certain times, intermittent infusion is used, which does not require additional fluid. It can use the same techniques as an intravenous drip (pump or gravity drip), but after the complete dose of medication has been given, the tubing is disconnected from the IV access device. Some medications are also given by IV push or bolus, meaning that a syringe is connected to the IV access device and the medication is injected directly (slowly, if it might irritate the vein or cause a too-rapid effect). Once a medicine has been injected into the fluid stream of the IV tubing there must be some means of ensuring that it gets from the tubing to the patient. Usually this is accomplished by allowing the fluid stream to flow normally and thereby carry the medicine into the bloodstream; however, a second fluid injection is sometimes used, a “flush”, following the injection to push the medicine into the bloodstream more quickly.

“Oral administration” is a route of administration where a substance is taken through the mouth, and includes buccal, sublabial and sublingual administration, as well as enteral administration and that through the respiratory tract, unless made through e.g. tubing so the medication is not in direct contact with any of the oral mucosa. Typical form for the oral administration of therapeutic agents includes the use of tablets or capsules.

The term “ranolazine” or “RAN” refers to the compound named “±-N-(2,6-dimethylphenyl)-4-[2-hydroxy-3-(2-methoxyphenoxy)-propyl]-1-piperazineacetamide,” and its pharmaceutically acceptable salts. Ranolazine is disclosed in U.S. Pat. No. 4,567,264 for use in the treatment of cardiovascular diseases, including arrhythmias, variant and exercise-induced angina, and myocardial infarction. Ranolazine is represented by the chemical formula:

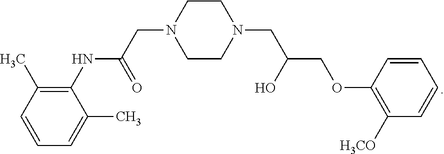 [SMILES: COc1ccccc1OCC(O)CN1CCN(CC(=O)Nc2c(C)cccc2C)CC1]

Compound A refers to 6-(4-(trifluoromethoxy)phenyl)-3-(trifluoromethyl)-[1,2,4]triazolo[4,3-α]pyridine and has a structure of:

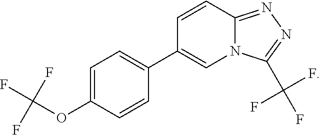

“Aminocarbonylmethyl” refers to a group having the following structure:

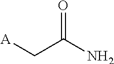

where A represents the point of attachment.

“Halo” or “halogen” refers to fluoro, chloro, bromo or iodo.

“Lower acyl” refers to a group having the following structure:

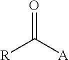

where R is lower alkyl as is defined herein, and A represents the point of attachment, and includes such groups as acetyl, propanoyl, n-butanoyl and the like.

“Lower alkyl” refers to an unbranched saturated hydrocarbon chain of 1-4 carbons, such as methyl, ethyl, n-propyl, and n-butyl.

“Lower alkoxy” refers to a group —OR wherein R is lower alkyl as herein defined.

“Lower alkylthio” refers to a group —SR wherein R is lower alkyl as herein defined.

“Lower alkyl sulfinyl” refers to a group of the formula:

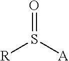

wherein R is lower alkyl as herein defined, and A represents the point of attachment.

“Lower alkyl sulfonyl” refers to a group of the formula:

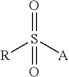

wherein R is lower alkyl as herein defined, and A represents the point of attachment.

“N-Optionally substituted alkylamido” refers to a group having the following structure:

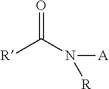

wherein R is independently hydrogen or lower alkyl and R′ is lower alkyl as defined herein, and A represents the point of attachment.

A compound of a given formula (e.g. the compound of Formula I) is intended to encompass the compounds of the disclosure, and the pharmaceutically acceptable salts, pharmaceutically acceptable esters, isomers, solvates, isotopes, hydrates, polymorphs, and prodrugs of such compounds. Additionally, the compounds of the disclosure may possess one or more asymmetric centers, and can be produced as a racemic mixture or as individual enantiomers or diastereoisomers. The number of stereoisomers present in any given compound of a given formula depends upon the number of asymmetric centers present (there are 2ⁿ stereoisomers possible where n is the number of asymmetric centers). The individual stereoisomers may be obtained by resolving a racemic or non-racemic mixture of an intermediate at some appropriate stage of the synthesis or by resolution of the compound by conventional means. The individual stereoisomers (including individual enantiomers and diastereoisomers) as well as racemic and non-racemic mixtures of stereoisomers are encompassed within the scope of the present disclosure, all of which are intended to be depicted by the structures of this specification unless otherwise specifically indicated.

“Isomers” are different compounds that have the same molecular formula. Isomers include stereoisomers, enantiomers and diastereomers.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(±)” is used to designate a racemic mixture where appropriate.

“Diastereoisomers” are stereoisomers that have at least two asymmetric atoms, but which are not mirror-images of each other.

The absolute stereochemistry is specified according to the Cahn Ingold Prelog R S system. When the compound is a pure enantiomer the stereochemistry at each chiral carbon may be specified by either R or S. Resolved compounds whose absolute configuration is unknown are designated (+) or (−) depending on the direction (dextro- or laevorotary) that they rotate the plane of polarized light at the wavelength of the sodium D line.

The term “polymorph” refers to different crystal structures of a crystalline compound. The different polymorphs may result from differences in crystal packing (packing polymorphism) or differences in packing between different conformers of the same molecule (conformational polymorphism).

The term “solvate” refers to a complex formed by the combining of a compound of Formula I, or any other formula as disclosed herein, and a solvent.

The term “hydrate” refers to the complex formed by the combining of a compound of Formula I, or any formula disclosed herein, and water.

The term “prodrug” refers to compounds of Formula I, or any formula disclosed herein, that include chemical groups which, in vivo, can be converted and/or can be split off from the remainder of the molecule to provide for the active drug, a pharmaceutically acceptable salt thereof or a biologically active metabolite thereof.

The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Amines are of general structure N(R³⁰)(R³¹)(R³²), wherein mono-substituted amines have 2 of the three substituents on nitrogen (R³⁰, R³¹ and R³²) as hydrogen, di-substituted amines have 1 of the three substituents on nitrogen (R³⁰, R³¹ and R³²) as hydrogen, whereas tri-substituted amines have none of the three substituents on nitrogen (R³⁰, R³¹ and R³²) as hydrogen. R³⁰, R³¹ and R³² are selected from a variety of substituents such as hydrogen, optionally substituted alkyl, aryl, heteroayl, cycloalkyl, cycloalkenyl, heterocyclyl and the like. The above-mentioned amines refer to the compounds wherein either one, two or three substituents on the nitrogen are as listed in the name. For example, the term “cycloalkenyl amine” refers to cycloalkenyl-NH₂, wherein “cycloalkenyl” is as defined herein. The term “diheteroarylamine” refers to NH(heteroaryl)₂, wherein “heteroaryl” is as defined herein and so on.

Specific examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

### 2. METHODS

Glucose homeostasis is regulated primarily by the opposing actions of insulin and glucagon secreted by pancreatic islets from beta- and alpha-cells, respectively. Various experimental studies have described an inhibitory effect of insulin and zinc released from β-cells on glucagon secretion. The number of β-cells is significantly reduced in T1 and T2DM which can result in loss of insulin-induced suppression of glucagon release by α-cells, and this may account for the hyperglucagonemia associated with T2DM.

Insufficient suppression of glucagon secretion post-prandially, as well as fasting hyperglucagonemia, have been observed in patients with diabetes. The elevated glucagon levels contribute to the hyperglycemia of type 2 diabetes by hepatic glucose output in both fasting and fed states. Therefore, it is contemplated that reduction of hyperglucagonemia by inhibiting glucagon secretion from α-cells improves glucose homeostasis.

Regulation of glucagon secretion is mediated by electrical machinery comprised of ion channels and paracrine factors. α-Cells contain a large tetrodotoxin (TTX)-sensitive Na⁺ current that inactivates at intermediate voltages, and plays a key role in glucagon secretion. It has been shown that α-cells of diabetic mice have upregulated glucagon content, express larger Na⁺ current and have increased action potential duration, amplitude and firing frequency as compared to cells from normal mice. These conditions sensitize the cells for increased glucagon secretion in response to low glucose.

In addition to insulin resistance and beta cell dysfunction, the pathophysiology of T2DM is characterized by hyperglucagonemia in the fasting state and lack of glucagon suppression following oral glucose, as well as exaggerated glucagon responses to mixed meal ingestion. During fasting conditions, hyperglucagonemia of T2DM sustains glucose overproduction in the liver, thus contributing significantly to fasting hyperglycemia. Similarly, exaggerated glucagon responses following ingestion of nutrients in T2DM result in inadequate suppression of high glucose production, thus contributing significantly to postprandial hyperglycemia. Therefore, reduction of glucagon hypersecretion can have a profound effect to mitigate hyperglycemia in T2DM.

The present disclosure demonstrates inhibition of sodium channels that are localized in the pancreas, and in particular those compounds that are selective inhibitors of tetrodotoxin (TTX)-s sodium channels, and are useful for treating diabetes and any other condition where glucagon secretion from alpha cells of the pancreas is too high. Thus the present disclosure also provides use of sodium-channel blockers for treatment of diabetes (T1 and 2) and related diseases where glucagon levels may be abnormally high.

The present disclosure demonstrates that sodium-channel blockers indeed inhibited glucagon secretion in pancreatic islets. Altogether, it is the present inventors' discovery that sodium-channel blockers provide a new approach for the treatment of hyperglycemia and related diseases and/or conditions, such as but not limited to, diabetes, elevated plasma level of HbA1c and elevated glucose plasma levels and may delay onset of diabetic complication in a diabetic or pre-diabetic.

One embodiment of the present disclosure provides a method of reducing the secretion of glucagon from an alpha cell, comprising contacting the alpha cell with an agent that suppresses the conduction of sodium ions through sodium channels. The contact can be in vivo, in vitro or ex vivo.

Another embodiment provides a method of lowering the plasma level of HbA1c, and/or glucose, delaying onset of diabetic complications, and/or treating diabetes in a patient having enhanced glucagon secretion compared to a normal individual, comprising administering to the patient an effective amount of an agent that suppresses the conduction of sodium ions through sodium channels.

Yet another embodiment provides a method of lowering the plasma level of HbA1c, and/or glucose, delaying onset of diabetic complications, and/or treating diabetes in a patient, comprising administering to the patient an effective amount of an agent that suppresses the conduction of sodium ions through sodium channels.

The treatment effect can be measured clinically. Plasma levels of HbA1c and glucose, for instance, can all be measured by blood test. Assessment of other symptoms of a diabetic patient, such as renal injury, is also within the knowledge of the skilled artisan.

A patient having elevated glucagon levels may be compared with a normal or healthy individual. Methods of measuring glucagon plasma levels are known in the art. See, e.g., Müller W A et al. “Abnormal alpha-cell function in diabetes. Response to carbohydrate and protein ingestion,” N Engl J. Med. 1970 Jul. 16; 283(3):109-15, Christensen M et al., “Glucose-dependent insulinotropic polypeptide: a bifunctional glucose-dependent regulator of glucagon and insulin secretion in humans,” Diabetes. 2011 December; 60(12):3103-9. Epub 2011 Oct. 7, Menge B A et al., “Loss of inverse relationship between pulsatile insulin and glucagon secretion in patients with type 2 diabetes,” Diabetes. 2011 August; 60(8):2160-8. Epub 2011 Jun. 15, Oskarsson P R et al., “Circulating insulin inhibits glucagon secretion induced by arginine in type 1 diabetes,” Eur J. Endocrinol. 2000 January; 142(1):30-4.

### 3. COMBINATION THERAPIES

The present inventors' discoveries demonstrate that that inhibition of abnormal glucagon secretion from α-cells by sodium-channel blockers are useful for the treatment of hyperglycemia and related diseases and conditions. A conventional treatment for hyperglycemia includes the injection or induced secretion of insulin or induction responses downstream of insulin. As insulin secretion and glucagon secretion are two separate processes, one by the β-cell and the other by the α-cell, it is contemplated that when two agents are given to a patient concurrently, a synergistic treatment effect ensues.

Accordingly, one embodiment of the present disclosure provides a method of treating diabetes in a patient, comprising administering to the subject (a) a synergistically therapeutically effective amount of insulin or a drug that increases the production of insulin or sensitivity to insulin and (b) a synergistically therapeutically effective amount of an agent that suppresses the conduction of sodium ions through sodium channels.

Drugs that increase the production of insulin or sensitivity to insulin are also known in the art. Non-limiting examples include a thiazolidinedione, a sulfonylurea, a meglitinide, an alpha-glucosidase inhibitor, an incretin mimetic, and an amylin analogue.

Non-limiting examples of drugs that increase the production of insulin or sensitivity to insulin include sulfonylureas (including chlorpropamide (Orinase®), tolbutamide (Tolinase®), glyburide (Micronase®), glipizide (Glucotrol®), and glimepiride (Amaryl®)) meglitinides (including reparglinide (Prandin®) and nateglinide (Starlix®)), and pioglitazone (Actos®). Methods of preparing fixed dose combination drugs (therapy) are known to one of skill in the art.

Depending on the formulation and designated administration route of the Na-channel blocker and the drug that increases the production of insulin or sensitivity to insulin (or insulin itself), how these drugs are administered to a patient can be determined by a competent caregiver. In one aspect, the administration is oral for both; in another aspect, one can be administered orally and the other injected; yet in another aspect, both are injected. Injection can be intravenous or intramuscular, without limitation.

In one aspect, the sodium-channel blocker is administered within a timeframe determined by a competent caregiver before insulin or the drug that increases the production of insulin or sensitivity to insulin. In another aspect, the Na-channel blocker is administered within a timeframe determined by a competent caregiver after insulin or the drug that increases the production of insulin or sensitivity to insulin. In yet another aspect, the Na-channel blocker is administered concurrently with insulin or the drug that increases the production of insulin or sensitivity to insulin.

Pursuant to the contemplated synergy and the combination treatment methods, the present disclosure further provides a composition, product, package or kit comprising (a) a synergistically therapeutically effective amount of insulin or a drug that increases the subject's production of insulin or sensitivity to insulin and (b) a synergistically therapeutically effective amount of an agent that suppresses the conduction of sodium ions through sodium channels. The compositions herein may be in the form of a fixed dose combination (a) and (b) or separate doses of (a) and (b).

Synergy between two different Na-channel blockers is also contemplated. In one aspect, one of the Na-channel blockers is ranolazine and the other is any Na-channel as disclosed herein. Accordingly, one embodiment of the present disclosure provides a composition, product, package or kit comprising a synergistically therapeutically effective amount of an agent that suppresses the conduction of sodium ions through sodium channels and a synergistically therapeutically effective amount of a different agent that suppresses the conduction of sodium ions through sodium channels.

In a preferred embodiment, said two or more sodium channel inhibitor compounds are delivered as a fixed dose combination.

### 4. NA-CHANNEL BLOCKERS

Various “agents that suppress the conduction of sodium (Na) ions through sodium channels” or “sodium (Na)-channel blockers” are known in the art.

For instance, alkaloid based toxins such as tetrodotoxin (TTX) and saxitoxin (STX) are substances that block sodium channels by binding to and occluding the extracellular pore opening of the channel.

Certain agents, on the other hand, block the sodium channels by blocking from the intracellular side of the channel. Such agents include, for instance, local anesthetics, Class I antiarrhythmic agents, and anticonvulsants.

Specific examples of sodium-channel blockers include ranolazine, lidocaine, mexiletine, flecamide, amiloride, triamterene, benzamil, A-803467, quinidine, procainamide, disopyramide, tocamide, phenyloin, encamide, moricizine, and propafenone.

Lidocaine, commercially available as Xylocalne® or lignocaine, is a sodium-channel blocker and local anesthetic and antiarrhythmic drug. Lidocaine is used topically to relieve itching, burning and pain from skin inflammations, injected as a dental anesthetic or as a local anesthetic for minor surgery.

Mexiletine, commercially available as Mexitil®, is a sodium-channel blocker and belongs to the Class IB anti-arrhythmic group of medicines. Mexiletine is used to treat arrhythmias within the heart, or seriously irregular heartbeats. Mexiletine slows conduction in the heart and makes the heart tissue less sensitive. Dizziness, heartburn, nausea, nervousness, trembling, unsteadiness are common side effects. Mexiletine is available in injection and capsule form.

Flecamide acetate is a sodium-channel blocker and a class Ic antiarrhythmic agent used to prevent and treat tachyarrhythmias (abnormal fast rhythms of the heart). It is also used to treat a variety of cardiac arrhythmias including paroxysmal atrial fibrillation (episodic irregular heartbeat originating in the upper chamber of the heart), paroxysmal supraventricular tachycardia (episodic rapid but regular heartbeat originating in the atrium), and ventricular tachycardia (rapid rhythms of the lower chambers of the heart). Flecamide works by regulating the flow of sodium in the heart, causing prolongation of the cardiac action potential.

Amiloride is a potassium-sparing diuretic, first approved for use in 1967 (then known as MK 870), used in the management of hypertension and congestive heart failure. Amiloride is a guanidinium group containing pyrazine derivative. Amiloride works by directly blocking the epithelial sodium channel (ENaC) thereby inhibiting sodium reabsorption in the late distal convoluted tubules, connecting tubules, and collecting ducts in the kidneys. This promotes the loss of sodium and water from the body, but without depleting potassium.

Triamterene, commercially available as Dyrenium®, is a potassium-sparing diuretic used in combination with thiazide diuretics for the treatment of hypertension and edema. Triamterene directly blocks the epithelial sodium channel (ENaC) on the lumen side of the kidney collecting tubule. Triamterene directly inhibits the entry of sodium into the sodium channels.

Benzamil, also known as “benzyl amiloride”, is a potent blocker of the ENaC channel and also a sodium-calcium exchange blocker. Benzamil is a potent analog of amiloride, and is marketed as the hydrochloride salt (benzamil hydrochloride).

A-803467: specific blockade of Naᵥ1.8 channels (SCN10A), developed by Icagen and Abbott Laboratories (see Jarvis et al., “A-803467, a potent and selective Nav1.8 sodium channel blocker, attenuates neuropathic and inflammatory pain in the rat,” PNAS 104 (20): 8520-5 (2007)).

Quinidine is a pharmaceutical agent that acts as a class I antiarrhythmic agent (Ia) in the heart. It is a stereoisomer of quinine, originally derived from the bark of the cinchona tree. The drug causes increased action potential duration, and well as a prolonged QT interval. Quinidine has a chemical name of “(9S)-6′-methoxycinchonan-9-ol” and CAS number 56-54-2.

Procainamide, also known as Pronestyl®, Procan® and Procanbid®, is a pharmaceutical antiarrhythmic agent used for the medical treatment of cardiac arrhythmias, classified by the Vaughan Williams classification system as class Ia. Procainamide has a chemical name of 4-amino-N-(2-diethylaminoethyl)benzamide and CAS of 51-06-9.

Disopyramide, also known as Norpace® and Rythmodan®, is an antiarrhythmic medication used in the treatment of Ventricular Tachycardia. Disopyramide is a sodium channel blocker and classified as a Class 1a anti-arrhythmic agent. Disopyramide also has an anticholinergic effect on the heart which accounts for many adverse side effects. Disopyramide has a chemical name of (RS)-4-(diisopropylamino)-2-phenyl-2-(pyridin-2-yl)butanamide, and CAS number 3737-09-05.

Tocamide is a lidocaine analog and is a class Ib antiarrhythmic agent. The chemical name of tocamide is N-(2,6-dimethylphenyl)alaninamide, with CAS number 41708-72-9.

Phenyloin sodium is a class 1b antiarrhythmic encamide. Phenyloin acts to suppress the abnormal brain activity seen in seizure by reducing electrical conductance among brain cells by stabilizing the inactive state of voltage-gated sodium channels. Aside from seizures, it is an option in the treatment of trigeminal neuralgia in the event that carbamazepine or other first-line treatment seems inappropriate. Phenyloin has a chemical name of 5,5-diphenylimidazolidine-2,4-dione and CAS number 57-41-0.

Moracizine, also known as Ethmozine®, is an antiarrhythmic of class IC. Moracizine was used for the prophylaxis and treatment of serious and life-threatening ventricular arrhythmias, but was withdrawn in 2007 for commercial reasons. The chemical name of moracizine is ethyl [10-(3-morpholin-4-ylpropanoyl)-10H-phenothiazin-2-yl]carbamate and the CAS number is 31883-05-3.

Propafenone, also known as Rythmol SR® and Rytmonorm®, is a class of anti-arrhythmic medication, which treats illnesses associated with rapid heart beats such as atrial and ventricular arrhythmias. The chemical name of propafenone is 1-{2-[2-hydroxy-3-(propylamino)propoxy]phenyl}-3-phenylpropan-1-one and the CAS number is 54063-53-5.

Other examples of sodium-channel blockers under development are shown in Table 1 below, indicating where (originator) the compounds may be obtained.

TABLE 1
Sodium-channel blockers under development

| Drug | Other Names | Originator | Ref. |
|---|---|---|---|
| A-76895 | IDDB2395 | Abbott | |
| | | Laboratories | |
| Nav1.7 inhibitor (pain) | | Amgen Inc | |
| AWD-33-173 | | ASTA Medica | WO-00007988 |
| | | AG | |
| LTA | 3737-39-1; sodium channel | AstraZeneca plc | |
| | blocker, AstraZeneca; | | |
| | AR-R-16444 | | |
| phenyl isoxazole | Nav1.7 inhibitor (pain), | AstraZeneca plc | |
| voltage-gated (Nav) Na+ | AstraZeneca; IDDBCP273585; | | |
| channel blockers | phenyl isoxazole voltage-gated | | |
| (neuropathic pain) | (Nav) Na+ channel blockers | | |
| | (neuropathic pain), AstraZeneca; | | |
| | AZ-1297442; voltage-gated Na+ | | |
| | channel subunit alpha inhibitor | | |
| | (pain), AstraZeneca | | |
| RPR-203484 | | Aventis Pharma | |
| | | SA | |
| Nav1.7 blockers (pain) | Axxam/Newron/Primm; voltage | Axxam SpA | |
| | gated sodium channel inhibitors | | |
| | (pain), Axxam/Newron/Primm | | |
| BAY-39-9437 | | Bayer AG | |
| BIA-2-024 | 199997-15-4; carbamazepine | BIAL Group | WO-09745416 |
| | analogs, BIAL; BIA-2-256; | | |
| | BIA-2-254; BIA-2-024 | | |
| eslicarbazepine acetate | 236395-14-5; Exalief; Stedesa; | BIAL Group | WO-09702250 |
| | Zebinix; SEP-0002093; | | |
| | 104746-04-5; eslicarbazepine; | | |
| | BIA-2-059; BIA-2-005; | | |
| | BIA-2-093 | | |
| crobenetine | 221018-88-8; BIII 890; | Boehringer | WO-09914199 |
| | 221019-25-6; BIII-890-CL | Ingelheim Corp | |
| BW-1003C87 | 144425-86-5 | Burroughs | |
| | | Wellcome Inc | |
| CNS-5151 | | CeNeS | |
| | | Pharmaceuticals | |
| | | Inc | |
| dual sodium/calcium | ion channel blockers (1), Scion; | CeNeS | |
| channel blockers (pain) | SPI-860; dual sodium/calcium | Pharmaceuticals | |
| | channel blockers (pain), Scion; | Inc | |
| | ion channel blockers, Cambridge | | |
| | NeuroScience; ion channel | | |
| | blockers (1), Scion/CeNeS | | |
| CEN-ep | | CenTRion | |
| | | Therapeutics Ltd | |
| CEN-ms | | CenTRion | |
| | | Therapeutics Ltd | |
| CEN-nep | | CenTRion | |
| | | Therapeutics Ltd | |
| CPL-7075 | mixed CB agonist/sodim | Cervelo | |
| | channel blocker (pain), Cervelo | Pharmaceuticals | |
| | | Ltd | |
| Nav1.7 inhibitors | SCN9A inhibitors (pain), | Chromocell | |
| | Chromocell; voltage-gated | Corporation | |
| | sodium channel 1.7 inhibitors | | |
| | (pain), Chromocell | | |
| DSP-2230 | | Dainippon | |
| | | Sumitomo | |
| | | Pharma Co Ltd | |
| E-2070 | sodium channel blocker | Eisai Co Ltd | |
| | (neuropathic pain), Eisai | | |
| ER-129517 | ER-129517 | Eisai Co Ltd | |
| neuron-specific calcium | 217170-95-1; calcium | Elan | |
| channel blockers | N-channel blockers, | Pharmaceuticals | |
| | Neurex/Warner; calcium | Inc | |
| | channel antagonist, Elan/Pfizer; | | |
| | omega conotoxin, Neurex; | | |
| | NSCC, Elan/Pfizer; PD-173212; | | |
| | PD-109084; PD-176078; | | |
| | PD-181283; PD-151307; | | |
| | PD-167341; PD-175069; | | |
| | 247130-18-3; 225925-12-2; | | |
| | 225925-09-7 | | |
| hydrocortisone + | 94-09-7; hydrocortisone acetate + | Embil | |
| benzocaine + bismuth | benzocaine + bismuth | Pharmaceutical | |
| subgallate + | subgallate + benzalkonium | | |
| benzalkonium chloride | chloride; 8001-54-5; 50-03-3; | | |
| | Kortos Cream; 99-26-3 | | |
| bidisomide | 116078-65-0; butanamide; | GD Searle & Co | |
| | SC-40230 | | |
| vinpocetine | 42971-09-5; apovincamine; | Gedeon Richter | US-04035370 |
| | Vinpocetine hydrochloride; | Ltd | |
| | TCV-3B; RGH-4405; Cavinton; | | |
| | 107316-99-4 | | |
| ICM-I-136 | sodium channel blocker | Georgetown | |
| | (cancer), Georgetown University | University | |
| 4030W92 | 189013-61-4; analgesic, Glaxo | Glaxo Wellcome | WO-00061231 |
| | Wellcome; GR-4030W92; | plc | |
| | BW-4030W92; GW-273227 | | |
| BW-202W92 | | Glaxo Wellcome | |
| | | plc | |
| BW-618C89 | | Glaxo Wellcome | |
| | | plc | |
| GW-286103 | GW-286103X | Glaxo Wellcome | |
| | | plc | |
| lamotrigine | 84057-84-1; Lamictal CD; | Glaxo Wellcome | EP-00021121 |
| | Labileno; BW-430C; 430078; | plc | |
| | Lamictal | | |
| sipatrigine | 130800-90-7; BW-619C; 619C; | Glaxo Wellcome | |
| | BW 619C89 mesylate; | plc | |
| | BW-619-C89; 619C89; | | |
| | 130801-14-8 | | |
| lamotrigine | 84057-84-1; Lamictal XR; | GlaxoSmithKline | EP-00021121 |
| | lamotrigine; Lamictal | plc | |
| berlafenone | 18965-97-4; Bipranol; GK-23G; | Helopharm | |
| | berlafenone | | |
| diprafenone | 81447-80-5; diprafenone; | Helopharm | |
| | butafenone | | |
| Hoe-694 | 149725-40-6; Hoe-694 | Hoechst AG | WO-00224637 |
| PF-05089771 | voltage-gated sodium channel | Icagen Inc | |
| | 1.7 blockers (pain), Pfizer; | | |
| | SCN9A blockers (pain), | | |
| | Pfizer/Icagen/Birkbeck; | | |
| | PF-05089771; Nav1.7 blockers | | |
| | (pain), Pfizer | | |
| transcainide | 88296-62-2; R-61748; | Janssen | |
| | transcainide; R-54718 | Pharmaceutica | |
| | | NV | |
| topiramate | 97240-79-4; JNS-019; Topina; | Johnson & | EP-00138441 |
| | RWJ-17021-000; Topimax; | Johnson | |
| | Topamax; KW-6485; TPM; | | |
| | RWJ-17021; McN-4853; | | |
| | topiramate | | |
| DCUKA | DCUKA | Lohocla Research | |
| | | Corp | |
| iodoamiloride | 60398-23-4; 6-iodoamiloride; | Merck & Co Inc | |
| | 6-IA; iodoamiloride | | |
| voltage-gated sodium | IDDBCP266079; | Merck & Co Inc | |
| channel blockers | IDDBCP266078; | | |
| | IDDBCP266076; | | |
| | IDDBCP240037; | | |
| | IDDBCP240033; Nav1.7 | | |
| | blockers (neuropathic pain), | | |
| | Merck & Co; IDDBCP214799; | | |
| | IDDBCP196027; | | |
| | IDDBCP181860 | | |
| PSD-509 | M-5004; endometriosis therapy | Metris | |
| | (intravaginal), Metris; | Therapeutics Ltd | |
| | endometriosis therapy | | |
| | (intravaginal), Plethora | | |
| | Solutions | | |
| sodium channel inhibitor | | MS Therapeutics | |
| | | Ltd | |
| Org-7797 | 80177-51-1 | MSD OSS BV | |
| Neu-P11 | Piromelatine; dual | Neurim | |
| | melatonin/serotonin agonists | Pharmaceuticals | |
| | (insomnia), Neurim | | |
| Neu-P12 | Nav1.7/Nav1.3 inhibitor (pain), | Neurim | |
| | Neurim | Pharmaceuticals | |
| dual voltage-gated sodium | Neuromed; Z-123212; Z-212; | Neuromed | |
| and calcium channel | dual voltage-gated sodium and | Pharmaceuticals | |
| modulators | calcium channel modulators | Inc | |
| | (pain), Zalicus; dual | | |
| | voltage-gated sodium and | | |
| | calcium channel modulators | | |
| | (pain), CombinatoRx; NP-A | | |
| NQ-1065 | small molecule therapeutics | NeuroQuest Inc | |
| | (neuropathic pain), NeuroQuest | | |
| NW-1063 | | Newron | |
| | | Pharmaceuticals | |
| | | SpA | |
| sodium channel blockers | sodium channel blockers | Newron | |
| | (pain/neuropathic pain), Newron | Pharmaceuticals | |
| | | SpA | |
| licarbazepine | 29331-92-8; GP-477901; | Novartis AG | WO-2004014391 |
| | TRI-477; TRI-447; MHD; | | |
| | GP-47779 | | |
| licarbazepine | 29331-92-8; LIC-477; | Novartis AG | WO-2005092294 |
| | licarbazepine; LIC-477D | | |
| oxcarbazepine | 28721-07-5; TRI-476; | Novartis AG | US-03642775 |
| | oxcarbamazepine; Trileptal; | | |
| | KIN-493; oxcarbazepine; | | |
| | GP-47680 | | |
| P-552-02 | P-552; sodium channel blocker | Parion Sciences | WO-03070182 |
| | (oral rinse, dry mouth in | Inc | |
| | Sjogrens disease), | | |
| | Parion/Kainos; PS-552-02; | | |
| | KM-552; 522-02; KM-003; | | |
| | CF-552; sodium channel blocker | | |
| | (oral, dry mouth/Sjogren's | | |
| | syndrome), Parion Sciences | | |
| PD-85639 | 149838-21-1 | Parke-Davis & Co | |
| Nav1.8 blockers | PF-01247324; IDDBCP234309; | Pfizer Inc | WO-2007056099 |
| | voltage-gated sodium channel | | |
| | 1.8 blockers (pain), Pfizer | | |
| U-54494A | 112465-94-8 | Pharmacia & | WO-08702584 |
| | | Upjohn Co | |
| Nav1.7 subunit sodium | | RaQualia Pharma | |
| channel blocker | | Inc | |
| Nav1.8 subunit sodium | Nav1.8 subunit sodium channel | RaQualia Pharma | |
| channel blocker | blocker | Inc | |
| RQ-00203066 | Nav1.3 antagonists (pain), | RaQualia Pharma | |
| | RaQualia | Inc | |
| lamotrigine + clonazepam | 1622-61-3; Cionamat; | RIMSA | |
| | lamotrigine + clonazepam | Laboratorios | |
| | (tablets, epilepsy/bipolar | | |
| | disease/restless legs syndrome), | | |
| | RIMSA/InterLab | | |
| | Pharmaceutica; 84057-84-1 | | |
| lifarizine | 119514-66-8; RS-87476-000; | Roche Bioscience | EP-00289227 |
| | RS-87476 | | |
| RS-100642 | 194027-17-3; RS-100642-198; | Roche Bioscience | |
| | 346670-94-8; 194027-14-0 | | |
| RS-2135 | 133775-36-7; (+)-RS-2135 | Sankyo Co Ltd | |
| dronedarone | 141626-36-0; Multac; Multaq; | Sanofi-Synthelabo | WO-2012076679 |
| | SR-35021; SR-33589 | | |
| SRSC-355 | SGX-211; neuropathic pain | Sirus | WO-2004014350 |
| | therapeutic (systemic depot, CK | Pharmaceuticals | |
| | polymer), Sirus; SGX-355 | Ltd | |
| ST-200 series | ST-2XX | SiteOne | |
| | | Therapeutics Inc | |
| topiramate | 97240-79-4; SRx-502; | Spherics Inc | |
| pilsicainide | 88069-67-4; Sunrhythm; | Suntory Ltd | EP-00089061 |
| | SUN-1165i; SUNRYTHM; | | |
| | DU-6552; SUN-1165; | | |
| | Pilsicainide hydrochloride; | | |
| | 88069-49-2 | | |
| CV-6402 | 118811-38-4 | Takeda | |
| | | Pharmaceutical | |
| | | Co Ltd | |
| T-477 | 136929-56-1 | Tanabe Seiyaku | |
| | | Co Ltd | |
| RSD-921 | 114419-77-1; PD 123497 | University of | |
| | | British Columbia | |
| sodium channel blockers | 181144-66-1; IDDBCP161265; | University of | WO-00061188 |
| | IDDBCP150202; V-201696; | Saskatchewan | |
| | IDDB16361-2; V-111662; | | |
| | V-102862; Co-102862 | | |
| 44-Bu | | University of | |
| | | Veterinary and | |
| | | Pharmaceutical | |
| | | Sciences Brno | |
| sodium channel | VRTX-C; VRTX-B; VRTX-A; | Vertex | WO-2006101629 |
| modulators | VX-409 | Pharmaceuticals | |
| | | Inc | |
| Tetrodin | Tetrodotoxin derivative (1), | WEX | WO-2005123088 |
| | Wex; Tetrodin (HT) | Pharmaceuticals | |
| | | Inc | |
| Tocudin | Tocudin; tetrodotoxin derivative | WEX | WO-2005123088 |
| | (3), Wex | Pharmaceuticals | |
| | | Inc | |
| TTX-9401 | TTX; TTX-9401; intractable | WEX | WO-2005123088 |
| | pain therapy, WEX; tetrodotoxin | Pharmaceuticals | |
| | derivative (2), WEX; Tectin | Inc | |
| recainam | 74738-24-2; Vanorm; | Wyeth | WO-08000151 |
| | 74752-07-1; recainam | | |
| | hydrochloride; Win-42362; | | |
| | Wy-42362 | | |
| NaV1.7 inhibitors | XEN-907 | Xenon | |
| | | Pharmaceuticals | |
| | | Inc | |
| XEN-402 | XEN-402; IDDBCP273282; | Xenon | WO-2006110917 |
| | analgesic (oral, pain), Xenon | Pharmaceuticals | |
| | Pharmaceuticals; XPF-001 | Inc | |
| Nav1.7 subunit sodium | SCN9A antagonists (oral, pain), | Zalicus Inc | |
| channel antagonists | Zalicus; voltage-gated sodium | | |
| | channel 1.7 antagonists (oral, | | |
| | pain), Zalicus | | |
| ZM-227189 | IDDB8206 | Zeneca Group plc | |
| AM-66 | pain therapeutic (sodium | Zenyth | WO-00236590 |
| | channel blocker), AMRAD | Therapeutics Ltd | |
| CNSB-002 | 199467-52-2; sodium channel | Zenyth | WO-09743259 |
| | antagonist (pain), Relevare; | Therapeutics Ltd | |
| | Brain injury therapy, AMRAD; | | |
| | Alzheimers therapy, AMRAD; | | |
| | AM-36 | | |

The above late sodium channel inhibitor compounds in various stages of development are contemplated to be useful for the practice of the technology disclosed herein. As such, it is contemplated that such compounds may be used alone singly or in combination with each other or with other therapies for diabetes and complications thereof disclosed herein, for the treatment of diabetes and complications thereof.

In some embodiments, the sodium-channel blocker is not a compound of Formula I as defined below:

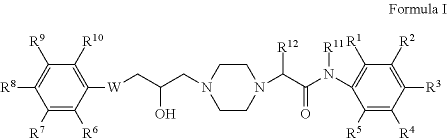

wherein:

R¹, R², R³, R⁴ and R⁵ are each independently hydrogen, lower alkyl, lower alkoxy, cyano, trifluoromethyl, halo, lower alkylthio, lower alkyl sulfinyl, lower alkyl sulfonyl, or N-optionally substituted alkylamido, provided that when R¹ is methyl, R⁴ is not methyl;

or R² and R³ together form —OCH₂O—;

R⁶, R⁷, R⁸, R⁹ and R¹⁰ are each independently hydrogen, lower acyl, aminocarbonylmethyl, cyano, lower alkyl, lower alkoxy, trifluoromethyl, halo, lower alkylthio, lower alkyl sulfinyl, lower alkyl sulfonyl, or di-lower alkyl amino; or

R⁶ and R⁷ together form —CH═CH—CH═CH—; or

R⁷ and R⁸ together form —O—CH₂O—;

R¹¹ and R¹² are each independently hydrogen or lower alkyl; and

W is oxygen or sulfur;

or a pharmaceutically acceptable salt, ester or prodrugs thereof, or an isomer thereof.

In one aspect, the sodium-channel blocker is not ranolazine.

The compounds of Formula I are disclosed in more detail in U.S. Pat. No. 4,567,264, the complete disclosure of which is hereby incorporated by reference.

### 5. PHARMACEUTICAL COMPOSITIONS AND ADMINISTRATION

The compositions, agents and drugs of the disclosure are usually administered in the form of pharmaceutical compositions. This disclosure therefore provides pharmaceutical compositions that contain, as the active ingredient, one or more of the compounds of the disclosure, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The agents of the disclosure may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G. S. Banker & C. T. Rhodes, Eds.).

The compositions of the disclosure may be administered in either single or multiple doses by any of the accepted modes of administration of the composition having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One preferred mode for administration is parental, particularly by injection. The forms in which the novel compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the disclosure in the required amount in the appropriate solvent with various other ingredients as enumerated above, as required, followed by filtration and sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration. Administration may be via tablet, capsule or enteric-coated tablets, or the like. In making the pharmaceutical compositions that include at least one agent, the active ingredient is usually diluted by an excipient and/or enclosed within a carrier such that the formulation can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the disclosure can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; 5,616,345; and WO 0013687. Another formulation for use in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human patients or other mammals, each unit containing a predetermined quantity of active material calculated to produce the desired therapeutic effect, in association with a suitable pharmaceutical excipient (e.g., a tablet, capsule, ampoule). The agents are effective over a wide dosage range and are generally administered in a pharmaceutically effective amount. Preferably, for oral administration, each dosage unit contains from 10 mg to 2 g of an agent, more preferably 10 to 1500 mg, more preferably from 10 to 1000 mg, more preferably from 10 to 700 mg, and for parenteral administration, preferably from 10 to 700 mg of the agent, more preferably about 50 to 200 mg. It will be understood, however, that the amount of the agent actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route, for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

Agents of the disclosure may be impregnated into a stent by diffusion, for example, or coated onto the stent such as in a gel form, for example, using procedures known to one of skill in the art in light of the present disclosure.

The sustained release formulations of this disclosure are preferably in the form of a compressed tablet comprising an intimate mixture of compound and a partially neutralized pH-dependent binder that controls the rate of dissolution in aqueous media across the range of pH in the stomach (typically approximately 2) and in the intestine (typically approximately about 5.5). An example of a sustained release formulation is disclosed in U.S. Pat. Nos. 6,303,607; 6,479,496; 6,369,062; and 6,525,057, the complete disclosures of which are hereby incorporated by reference.

To provide for a sustained release of a compound, one or more pH-dependent binders are chosen to control the dissolution profile of the compound so that the formulation releases the drug slowly and continuously as the formulation passed through the stomach and gastrointestinal tract. The dissolution control capacity of the pH-dependent binder(s) is particularly important in a sustained release formulation because a sustained release formulation that contains sufficient compound for twice daily administration may cause untoward side effects if the compound is released too rapidly (“dose-dumping”).

Accordingly, the pH-dependent binders suitable for use in this disclosure are those which inhibit rapid release of drug from a tablet during its residence in the stomach (where the pH is below about 4.5), and which promotes the release of a therapeutic amount of compound from the dosage form in the lower gastrointestinal tract (where the pH is generally greater than about 4.5). Many materials known in the pharmaceutical art as “enteric” binders and coating agents have the desired pH dissolution properties. These include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof.

Preferred pH-dependent binder materials that can be used in conjunction with the compound to create a sustained release formulation are methacrylic acid copolymers. Methacrylic acid copolymers are copolymers of methacrylic acid with neutral acrylate or methacrylate esters such as ethyl acrylate or methyl methacrylate. A most preferred copolymer is methacrylic acid copolymer, Type C, USP (which is a copolymer of methacrylic acid and ethyl acrylate having between 46.0% and 50.6% methacrylic acid units). Such a copolymer is commercially available, from Röhm Pharma as Eudragit® L 100-55 (as a powder) or L30D-55 (as a 30% dispersion in water). Other pH-dependent binder materials which may be used alone or in combination in a sustained release formulation dosage form include hydroxypropyl cellulose phthalate, hydroxypropyl methylcellulose phthalate, cellulose acetate phthalate, polyvinylacetate phthalate, polyvinylpyrrolidone phthalate, and the like.

One or more pH-independent binders may be in used in sustained release formulations in oral dosage forms. It is to be noted that pH-dependent binders and viscosity enhancing agents such as hydroxypropyl methylcellulose, hydroxypropyl cellulose, methylcellulose, polyvinylpyrrolidone, neutral poly(meth)acrylate esters, and the like, may not themselves provide the required dissolution control provided by the identified pH-dependent binders. The pH-independent binders may be present in the formulation of this disclosure in an amount ranging from about 1 to about 10 wt %, and preferably in amount ranging from about 1 to about 3 wt % and most preferably about 2.0 wt %.

### EXAMPLES

The following examples are included to demonstrate embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

Unless otherwise stated all temperatures are in degrees Celsius. Also, in these examples and elsewhere, abbreviations have the following meanings:

| | |
|---|---|
| ATCC = | American Type Culture Collection |
| BSA = | bovine serum albumin |
| DMEM = | Dulbecco's modified Eagle's medium |
| DMSO = | dimethyl sulfoxide |
| DPBS = | Dulbecco's phosphate-buffered saline |
| ELISA = | Enzyme-linked immunosorbent assay |
| FBS = | fetal bovine serum |
| FPG = | fasting plasma glucose |
| HBSS = | hanks Balanced Salt solution |
| HEPES = | 4-(2-hydroxyethyl)-1-piperazineethanesulfonic |
| | acid |
| hr = | hour |
| IV = | intravenous |
| kg = | kilogram |
| M = | molar |
| mg = | milligram |
| mg/kg = | milligram/kilogram |
| mg/mL = | milligram/milliliter |
| min = | minute |
| mL = | milliliter |
| mM = | millimolar |
| NFG = | normal fasting glucose |
| nM = | nanomolar |
| PO = | oral |
| s = | second |
| STZ = | streptozotocin |
| TTX = | tetrodotoxin |
| U/mL = | units/milliliter |
| ZDF = | zucker diabetic fatty |
| μL or uL = | microliter |
| μM = | micromolar |
| μg = | microgram |

### Materials and Methods

### Isolation of Pancreatic Islets, Culture and Treatment

Pancreatic islets were isolated from male Sprague Dawley rats (8-12 weeks old, Charles River Laboratories Inc., Wilmington, Mass.). Briefly, Hanks Balanced Salt solution (HBSS) containing 0.3 mg/mL Liberase TL (Roche Diagnostics, Dallas), 0.12 mg/mL DNase I and 25 mM HEPES was infused into the pancreas of an anesthetized rat. The inflated pancreas was excised and incubated for 10 min at 37° C. Digestion was stopped by adding ice-cold Wash Buffer (HBSS with 5% FBS) and the tissue was pelleted by centrifugation at 450×g. Tissue pellets were resuspended with Wash Buffer, filtered through a 300 μm Nylon Mesh, and centrifuged at 450×g. Pancreatic islets were then purified by gradient centrifugation at 750×g with 4 different densities of islet gradient solutions (in the order of 1.108, 1.096, 1.06, and 1.037 g/mL, Mediatech, Inc). Islets were then collected from the interface of 1.096 and 1.06 g/mL gradient solutions and washed once with Wash Buffer by centrifugation at 450×g. Pellets of pancreatic islets were resuspended in islet culture medium (RPMI1640 containing 10% fetal bovine serum (FBS), 11 mM glucose, 100 U/mL penicillin, 100 μg/mL streptomycin, 2 mM L-glutamine, 10 mM HEPES, 1 mM sodium pyruvate), and cultured at 37° C. in 5% CO₂ for 1-4 days before experiments. Adult human pancreatic islets were obtained from National Disease Research Interchange and cultured 1-7 days before experiments.

Isolated rat or human islets with equal size were hand-picked under microscope and transferred to a 96-well plate with 10 islets per group in 200 μA of islet culture medium. Islets were then washed once with Krebs-Ringer buffer (129 mM NaCl, 4.8 mM KCl, 2.5 mM CaCl₂, 1.2 mM MgSO₄, 1.2 mM KH₂PO₄, 5 mM NaHCO3, and 10 mM HEPES, pH7.4) containing 0.1% BSA (fatty acid free) and 6 mM glucose, and then treated as indicated in 150 μl of Krebs-Ringer buffer containing 0.1% BSA and 3 mM glucose for 1 h at 37° C. in CO₂ incubator. Supernatants were harvested and stored at −80° C. until analysis. Glucagon levels were measured by an ELISA kit (BD biosciences, San Jose, Calif.).

### Culture and Treatment of α-TC1 Clone 9 Cells

α-TC1 clone 9 cells (obtained from ATCC) were cultured in DMEM medium supplemented with 16.5 mM glucose, 10% FBS, 100 U/mL penicillin, 100 μg/mL streptomycin, 2 mM L-glutamine, 15 mM HEPES, 1.5 g/L sodium bicarbonate, 0.1 mM non-essential amino acid, and subcultured every 3-4 days. Cells were seeded at 0.4×10⁵/well in 96-well plates and allowed to recover for 1 day. The media was then changed to serum-free DMEM and incubated overnight. Cells were then treated with sodium-channel blockers in the presence of veratridine (15 μM) in Krebs-Ringer buffer containing 0.1% BSA and 3 mM glucose for 1 h. Supernatants were collected and stored at −80° C. until analysis. Glucagon levels were measured by an ELISA kit.

### Dispersion and Culture of Pancreatic Alpha (α) Cells

Acutely isolated pancreatic islets were allowed to recover overnight at 37° C./5% CO₂ in Islet Media (RMPI 1640 supplemented with 10 mM HEPES, 1 mM sodium pyruvate, 10% FBS, 100 U/mL penicillin, 100 μg/mL streptomycin, 2 mM L-glutamine). The islets were resuspended and then centrifuged for 3 minutes at 200×g. The supernatant was discarded and 20 mL of filtered DPBS-EDTA was added (DPBS without Ca and Mg, 3 mM EDTA (G Biosciences), 0.5% BSA (Sigma), 1.5 mM dextrose (Sigma). The islets were incubated for 3 minutes at 37° C./5% CO₂ and then centrifuged for 3 minutes at 200×g. The pellet was resuspended in 5 mL of pre-warmed Accutase (Sigma) and transferred into a 60 mm suspension culture dish and incubated for 3 minutes at 37° C./5% CO₂. The digested islets were centrifuged for 3 minutes at 200×g to remove the accutase and the pellet was resuspended in 20 mL of room temperature DPBS-EDTA as defined above but with BSA increased to 4%. The digested islets were gently triturated 10 times with a flamed, glass Pasteur pipette and the suspension was then applied to a 40 μM cell strainer. The resulting single cell suspension was centrifuged for 3 minutes at 200×g and the cells were resuspended in 2 mL of pre-warmed Islet Media. The number of live cells was counted and diluted to 1×10⁵ with Islet Media. The cell suspension (2 mL) was added to a 35 mm cell culture dish containing PDL/Laminin coated coverslips (BD Biocoat) and incubated at 37° C./5% CO₂. The Islet Media was replaced (50%) after 6 hours to remove unattached cellular debris. Unless otherwise noted, all cell culture reagents were purchased from CellGro.

### Electrophysiological Measurements

Membrane potential and ion channel currents were recorded 24-72 hours after dispersion using the perforated patch configuration. The bath solution contained (in mM): 140 NaCl, 5 HEPES, 3.6 KCl, 2 NaHCO₃, 0.5 NaH₂PO₄, 0.5 MgSO₄, 2.6 CaCl₂, 10 dextrose, 10 sucrose with a pH adjusted to 7.35 with NaOH. Pipettes (3.5-5.0 MOhm) were pulled from borosilicate glass and tip-filled with an internal solution consisting of (in mM): 76 K₂SO₄, 10 KCl, 10 NaCl, 5 HEPES, 1 MgCl₂ with the pH adjusted to 7.35 with KOH. The pipette was back-filled with the intracellular solution supplemented with Amphotericin B (0.3 mg/mL) which provides low resistance perforated-patch access to the intracellular space. After forming the cell attached configuration, Amphotericin B diffusion into the patch was complete within 5 minutes. Series resistance (Rs) was monitored using a voltage step from −70 mV to 0 mV (5 ms, 0.5 Hz) and was allowed to stabilize prior to beginning the experiment (Rs<30 MOhm).

The identity of the α-cell was confirmed using cell size (membrane capacitance <6 pF) and the presence of electrical activity in low (3 mM) extracellular glucose, which are hallmarks of dispersed α-cells. Cells exhibiting spontaneous activity in 3 mM glucose were used for analysis. Cells exhibiting spontaneous activity in 10 mM glucose with no activity in 3 mM glucose illustrate the typical response of pancreatic β-cells and were excluded from analysis. Cells that did not exhibit spontaneous activity were excluded from analysis. Prior to recording membrane potential or ionic currents the series resistance was compensated to minimize recording artifacts. All recordings were made using pClamp 10.2 and were analyzed using Microsoft Excel 2003, Graphpad Prism7 or OriginPro7.

### Membrane Potential Recording

Recordings of membrane potential were made at 32° C. Recordings were made using a 200B Axopatch amplifier in I=0 current clamp mode with a low pass filter of 5 kHz and digitized at 10 kHz using an 1322A Digidata. Pipette resistance was compensated to minimize the response time of the signal. All drugs were dissolved in the bath solution and applied by bath exchange. For compounds dissolved in DMSO, the final concentration was 0.1% in all solutions, including the drug free solution. Representative records (30 sec) were analyzed using the event detection feature (threshold of 10 mV) to quantitate the spontaneous firing frequency and total charge movement. The membrane potential between events was measured for changes induced by the compound. Results are presented as mean±SEM.

### Ionic Current Recording

Recordings of ionic currents were made at 32° C. Recordings were made using a 200B Axopatch amplifier in voltage-clamp mode with a low pass filter of 5 kHz and digitized at 50 kHz using a 1322A Digidata. Series resistance was compensated to minimize voltage drop, charging time and filtering artifacts. Following confirmation of α-cell identity, the bath solution was changed to I_{Na}-bath in order to isolate the sodium current. The I_{Na}-bath solution contained (in mM): 130 NaCl, 5 HEPES, 3.6 KCl, 2 NaHCO₃, 0.5 NaH₂PO₄, 0.5 MgSO₄, 2.6CaCl₂, 3 dextrose, 20 TEA-Cl, 10 4-AP, 2.5 CoCl₂, 0.5 tolbutamide. The pH was first adjusted to 7.1 with HCl to dissolve the salts and then to 7.35 with NaOH. Leak currents were subtracted by using an online P/4 procedure. I_{Na} was measured using a voltage step to 0 mV (20 ms, 0.2 Hz) from a holding potential of either −70 mV or −90 mV. All drugs were dissolved in the I_{Na}-bath solution and applied by bath exchange. For compounds dissolved in DMSO, the final concentration was 0.1% in all solutions, including the drug free solution. The peak I_{Na} averaged over multiple sweeps was analyzed before and after application of the compound.

Expression of Human SCN3A (hNaV1.3) cDNA

HEK-293 cells stably expressing hNa_{V}1.23 (SCN3A NCBI #NM_{—}001081676.1, SCN1B NCBI #NM_{—}001037.4, SCN2B NCBI #NM_{—}004588.2) were obtained from Alfred George, Jr. (Vanderbilt University, Nashville, Tenn.). The cells were continuously maintained in a humidified, 5% CO₂ atmosphere at 37° C. in DMEM high glucose growth medium supplemented with 10% FBS, 2 mM L-glutamine, 100 U/mL penicillin, 100 μg/mL streptomycin, 1 mg/mL G418 and 3 μg/mL puromycin.

Expression of Human SCN9A (hNaV1.7) cDNA

HEK-293 cells stably expressing hNa_{V}1.7 (SCN9A, NCBI #NM_{—}002977 and SCN1B) were obtained from Scottish Biomedical (Glasgow, UK). The cells were continuously maintained in a humidified, 5% CO₂ atmosphere at 37° C. in MEM growth medium supplemented with 10% FBS, 2 mM L-glutamine, 100 U/mL penicillin, 100 μg/mL streptomycin, 0.6 mg/mL G418 and 2 μg/mL blastocydin.

### Automated Electrophysiology Recordings

Whole-cell voltage-clamp recordings were used to measure the activity of test compounds against hNa_{V}1.3 and hNa_{V}1.7. Sodium currents were recorded at room temperature using a QPatch 16× automated electrophysiological system (Sophion Bioscience, Copenhagen, Denmark). Cells were washed with PBS and then incubated with Detachin for 2 minutes at room temperature. Cells were then resuspended in growth media, pelleted by centrifugation and resuspended in CHO-S-SFM II serum free media supplemented with 100 U/mL penicillin, 100 μg/mL streptomycin and 10 mM HEPES. The cells were allowed to recover for 30 min at room temperature with constant stirring and then loaded into the QPatch cell holder.

The internal solution consisted of (in mM) 110 CsF, 10 NaF, 20 CsCl, 2 EGTA, 10 HEPES, with a pH of 7.35 and osmolarity of 300 mOsmol/kg. The external (control) solution contained in (mM): 145 NaCl, 4 KCl, 1.8 CaCl₂, 1 MgCl₂, 10 dextrose, 10 HEPES, with a pH of 7.35 and osmolarity of 310 mOsmol/kg. The cells were loaded into primed single hole (2 MΩ) large QPlates and the whole-cell configuration was established using the default protocol. Cells were allowed to stabilize for 10 min after establishment of the whole-cell configuration before current was measured. Series resistance was compensated (100%, τ=199 μs) to minimize voltage error and filtering artifacts. All currents are low-pass Bessel filtered at 5 kHz and digitized at 50 kHz.

Specific voltage-clamp protocols assessing voltage-dependent block (VDB, also termed state-dependent block) are used. VDB of peak current was measured following an 8 s conditioning prepulse (to -55 mV for Na_{V}1.3 and to −60 mV for Na_{V}1.7) followed by a test pulse to 0 mV (20 ms). An interpulse (−120 mV, 10 ms) was used to recover non-drug bound channels from fast inactivation. The prepulse potential was determined by the steady-state inactivation curves for Na_{V}1.3 and Na_{V}1.7. The VDB protocol was repeated at a frequency of 0.05 Hz. Multiple applications of drug were used to ensure complete solution exchange and the cells were allowed to stabilize in the drug containing solution for two minutes. Currents were normalized to the peak current recorded in the absence of drug and expressed as percent inhibition. Data analysis is performed Excel 2002 (Microsoft, Seattle, Wash., U.S.A.), and OriginPro 7.0 (OriginLab, Northampton, Mass., U.S.A) software.

Quantitative Real-Time RT-PCR (qPCR)

Total RNA was extracted from isolated pancreatic islets using TRIzol reagent (Invitrogen). cDNA was then synthesized using an iScript Reverse Transcription kit (BioRad, Hercules, Calif.). Primers used for qPCR are shown in Table 2. qPCR was performed using SYBR Green PCR reagents (Applied Biosystems, Foster City, Calif.) on Stratagene Mx3000P (Agilent, Santa Clara, Calif.). Relative mRNA levels were calculated by the delta Cₜ values (threshold cycle time) and normalized by the levels of β-actin.

TABLE 2
qPCR primer sequences

| | Forward primers (5′-3′) | Reverse primers (5′-3′) |
|---|---|---|
| Gene | (SEQ ID NO.) | (SEQ ID NO.) |
| human Nav1.1 | AGTCAATTACATCAGGACATTT (1) | GCAGTTCACGAATACAGTT (2) |
| human Nav1.2 | TGGCACTAGAACTGTATCA (3) | TGTAACTGGTAATATAACTTCACT (4) |
| human Nav1.3 | AACCCTGTCTCTCAAATG (5) | GGCACATAACTGTTCAGA (6) |
| human Nav1.4 | TACTCAGGGCATTCTGTT (7) | ACACTCAAGCACACATAC (8) |
| human Nav1.5 | CTCCTGTATCCTGTATCAATCTA (9) | TTGGCTTTTGTCATTTCCTT (10) |
| human Nav1.6 | ACAACCAACTAATTGACTA (11) | GGCTGTATGTTAGAGATG (12) |
| human Nav1.7 | CATCTTAGGTTCATTCATCTTAGG (13) | GGCTTGGTAGGTATGTGATAA (14) |
| human Nav1.8 | AATCTGAAACTGCTTCTG (15) | GTCCTCATGTTGACTCTA (16) |
| human Nav1.9 | TTCTGAGGATCTGTGGCTTGT (17) | TCTGGAAAGATTACTGGGTAGCA (18) |
| human β-actin | TGTACGCCAACACAGTGCTG (19) | CCGATCCACACGGAGTACTTG (20) |
| rat Nav1.1 | GTGCGTGTGTTTGTGTAC (21) | GCAGTAAGGAACAACATCTC (22) |
| rat Nav1.2 | TTTATTTCAGCACTTTCTTACG (23) | TTCCTGTTTGGGTCTCTTAG (24) |
| rat Nav1.3 | ACCGTCCATTCTAACCATC (25) | CGATAGCAGCAAGAGATTC (26) |
| rat Nav1.4 | CGCTCTTCTCTGCTTCTG (27) | CAATAGATTGTGCCACCTTC (28) |
| rat Nav1.5 | CACCTTCACTGCCATCTACAC (29) | TGCGTAAGGCTGAGACATTG (30) |
| rat Nav1.6 | TGATGATCCTGAC (31) | GCTCTCGTTGAAGTTTATGG (32) |
| rat Nav1.7 | CTGCTGAGAGTGAAGAAGAATTG (33) | GCTCGTGTAGCCATAATCCG (34) |
| rat Nav1.8 | GCATCAGGAACGGAACAG (35) | AGTGACCAGCATCAGACC (36) |
| rat Nav1.9 | CTTCACTTCCGACTCTCTG (37) | GCTTAGGTAACTTCCTGGAG (38) |
| rat β-actin | TTCAACACCCCAGCCATGT (39) | AGTGGTACGACCAGAGGCATACA (40) |

### Results

### 1. Effects of Sodium (Na)-Channel Blockers on Glucagon Secretion In-Vitro

### 1.1. Effects of Sodium (Na)-Channel Blockers on Glucagon Secretion in Rat and Human Pancreatic Islets Under Low Glucose Conditions

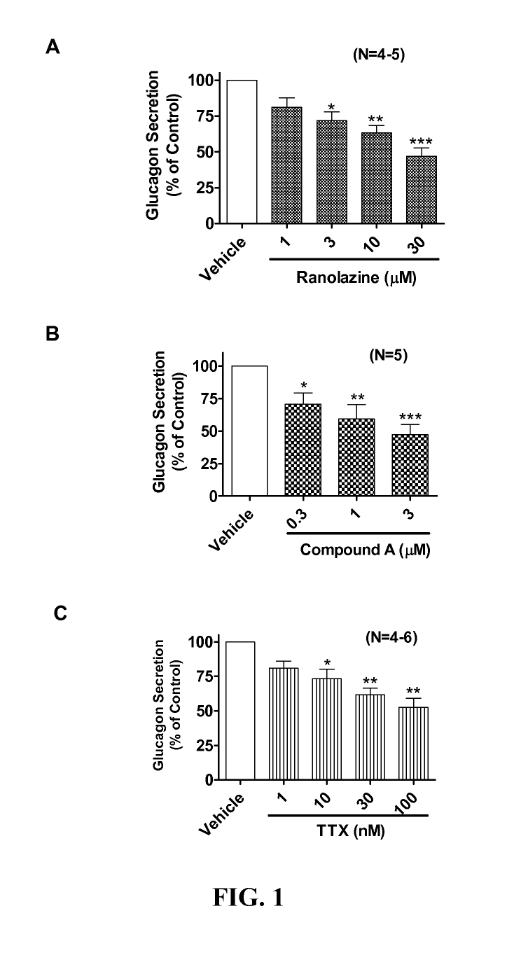

Pancreatic islets isolated from male Sprague-Dawley rats were used to determine the effects of various sodium-channel blockers (ranolazine, compound A and tetrodotoxin (TTX, a potent and selective sodium-channel blocker)) on glucagon secretion. All sodium-channel blockers significantly and concentration-dependently reduced glucagon secretion in the presence of 3 mM glucose (FIG. 1). As compared to the vehicle control, the maximal reduction of glucagon secretion was observed with ranolazine at 30 μM (53±6%), compound A at 3 μM (53±8%) and TTX at 100 nM (47±6%).

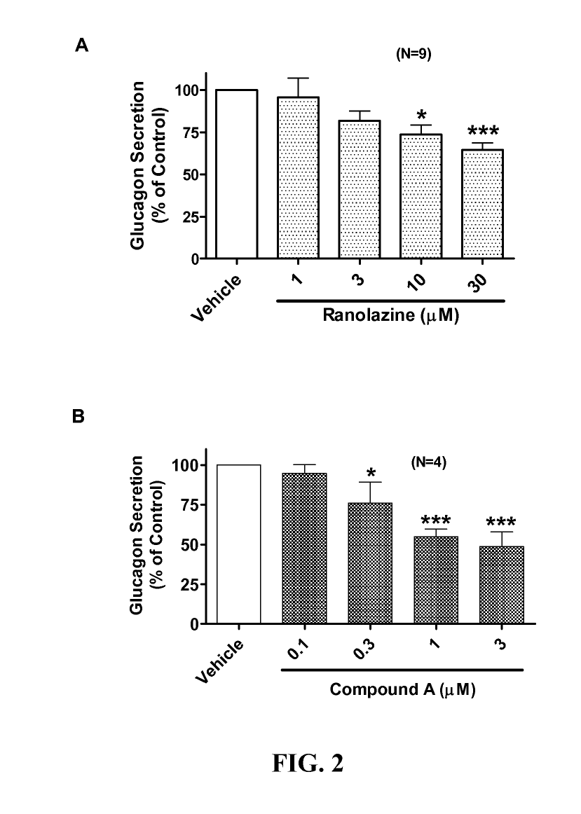

Similar to data in FIG. 1, the effects of sodium-channel blockers on glucagon secretion in human pancreatic islets (obtained from National Disease Research Interchange) were also determined. All sodium-channel blockers significantly and concentration-dependently reduced glucagon secretion from human pancreatic islets in the presence of 3 mM glucose (FIG. 2). As compared to the vehicle control, the maximal reduction of glucagon secretion was observed with ranolazine at 30 μM (36±4%), and compound A at 3 μM (51±9%).

### 1.2. Effects of Sodium Channel Blockers on Veratridine-Induced Glucagon Secretion in Rat and Human Pancreatic Islets

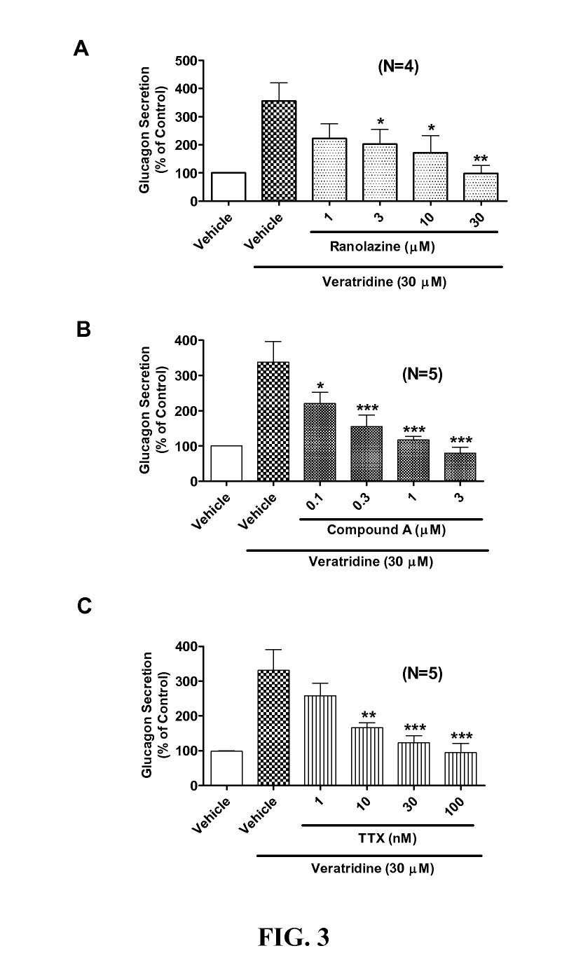

Veratridine, a sodium-channel activator (opener), increased glucagon secretion in a concentration-dependent manner suggesting that Na channels play a significant role in glucagon secretion in pancreatic islets. Veratridine at 30 μM caused more than 3-fold increase in glucagon secretion in rat pancreatic islets (FIG. 3). Ranolazine and other sodium-channel blockers significantly and concentration-dependently reduced the veratridine-induced increase in glucagon secretion. Complete reduction of veratridine-induced increase in glucagon secretion was observed for sodium-channel blockers at the highest concentration used for each compound.

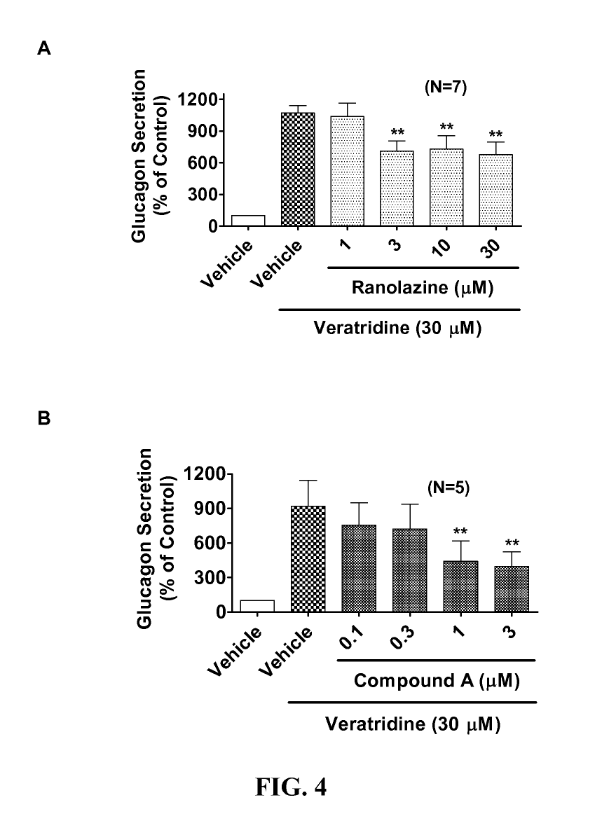

Similar data was obtained with various sodium-channel blockers on veratridine-induced glucagon secretion in human pancreatic islets (FIG. 4). Veratridine (30 μM) induced ˜9-fold increase in glucagon secretion in human pancreatic islets. All sodium-channel blockers significantly and concentration-dependently reduced veratridine-induced increase in glucagon secretion in human pancreatic islets (FIG. 4). Ranolazine, and compound A reduced veratridine-induced glucagon secretion by 36±9% at 30 μM and 58±7% at 3 μM respectively.

### 1.3. Effects of Sodium Channel Blockers on Veratridine-Induced Glucagon Secretion in α-TC1 Clone 9 Cells

Although glucagon is only produced by the α-cell, pancreatic islets contain several other cells types including the β-cell (insulin releasing) and δ-cell (somatostatin releasing) which can influence glucagon secretion. The reduction of glucagon release from intact islets could be secondary to a direct action of the tested sodium-channel blockers on other cell types. Therefore, the inhibition of glucagon release by sodium-channel blockers was investigated using a clonal α-cell (cell line). This experimental model removes any influence the other pancreatic cell types (paracrine signaling).

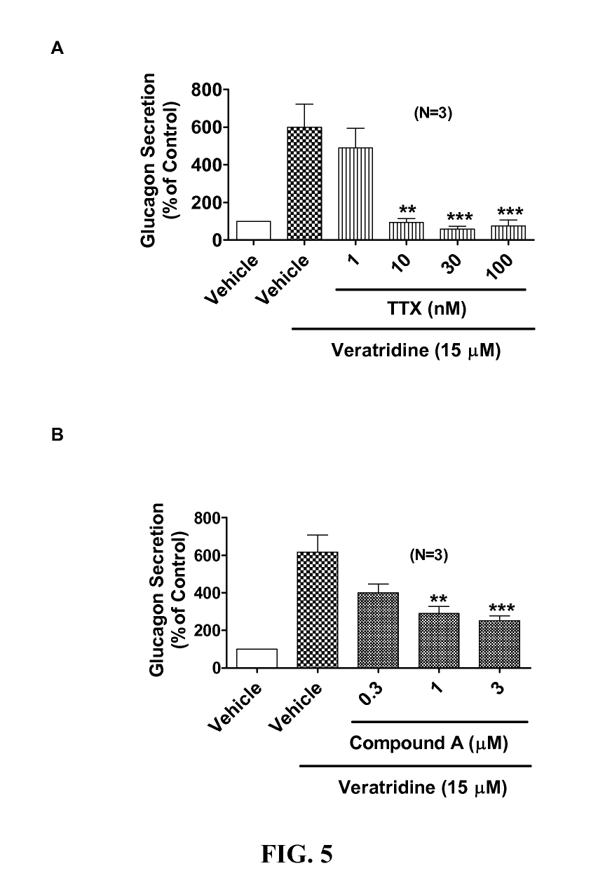

Similar to the effects on rat and human pancreatic islets, the sodium-channel blockers TTX and compound A significantly and concentration-dependently reduced veratridine (15 μM)-induced increase of glucagon secretion in α-TC1 clone 9 cells, by 100±6% at 100 nM and 70±4% at 3 μM respectively (FIG. 5).

### 1.4. Effects of Sodium Channel Blockers on Epinephrine- and Arginine-Induced Glucagon Secretion in Rat Pancreatic Islets

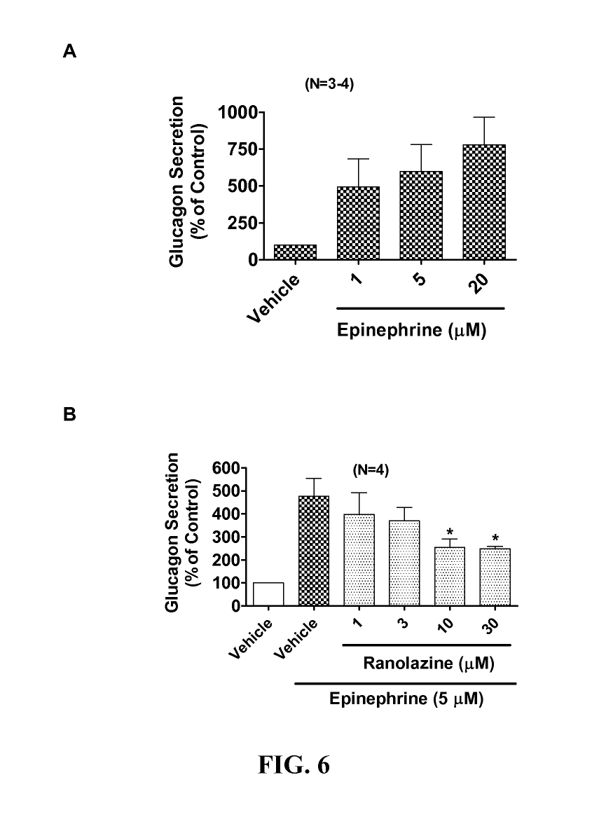

Glucagon secretion is affected by several physiological factors which include hormones and nutrients. Effect of sodium-channel blockers on glucagon secretion in response to sympathetic stimulation (epinephrine) and nutrients (arginine) was determined in rat pancreatic islets. Epinephrine increased glucagon secretion from rat pancreatic islets in a concentration-dependent manner (FIG. 6). Sodium-channel blocker ranolazine significantly and concentration-dependently reduced epinephrine (5 μM)-induced increase of glucagon secretion by 44±8% at 30 μM.

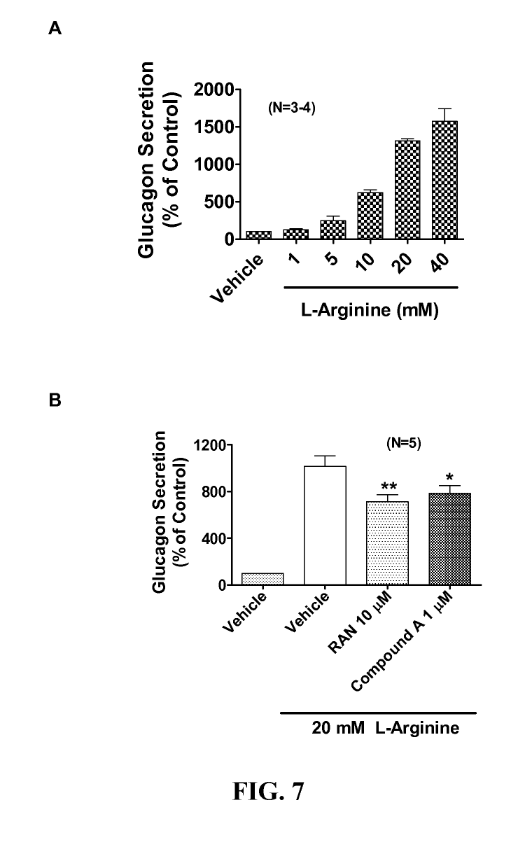

FIG. 7 shows the effects of sodium-channel blockers on the arginine-induced increase of glucagon secretion in rat pancreatic islets. L-arginine significantly increased glucagon secretion in rat pancreatic islets in a concentration-dependent manner. Sodium channel blockers ranolazine and compound A significantly reduced L-arginine (20 mM)-induced increase in glucagon secretion by 31±9% at 10 μM and 24±6% at 1 μM, respectively.

### 2. Effects of Sodium-Channel Blockers on Electrical Activity of Pancreatic α-Cells

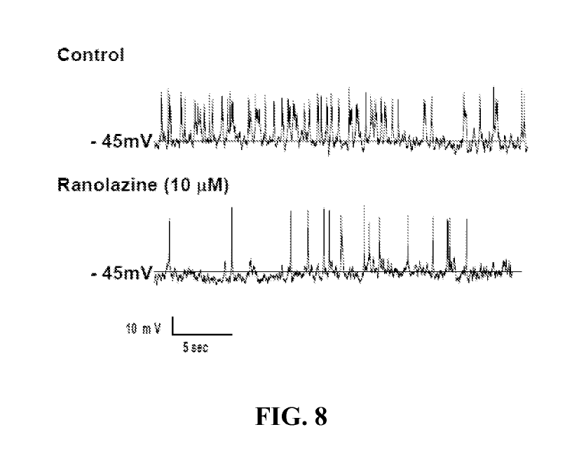

The spontaneous electrical activity (FIG. 8, upper panel, control) was reduced in the presence of 10 μM ranolazine (FIG. 8, lower panel, ranolazine) by 44%. Compound A reduced the spontaneous electrical activity of α-cells by 75% (at 0.3 μM) y.

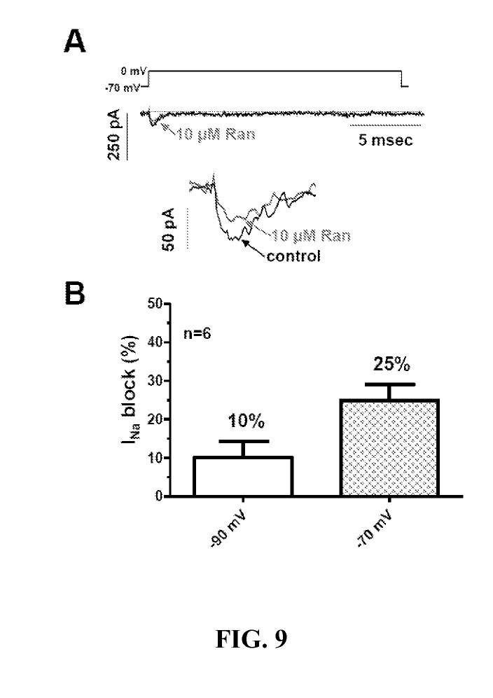

Peak Na⁺ current (I_{Na}) was recorded in isolated α-cells using Amphotericin-B (perforated) patch-clamp technique at 32° C. As shown in FIG. 9, rat isolated pancreatic α-cells were depolarized from a holding potential of −90 or -70 mV to 0 mV to record peak I_{Na}. Ranolazine caused a voltage-dependent block of peak I_{Na} at 10 μM by 10 and 25% at −90 and −70 mV, respectively (FIG. 9B). Compound A caused a 40 block of peak I_{Na} at 1 μM at −90 and -70 mV.

### 3. Anti-Diabetic Effects of Sodium-Channel Blockers In Vivo

### 3.1. Anti-Diabetic Effects of Ranolazine in Stz-Induced Diabetic Mouse, an Animal Model of Type 1 Diabetes

Streptozotocin (STZ) induces diabetes by selectively destroying the pancreatic β-cells. Five-week old male C57BL/6J mice were injected with STZ (40 mg/kg, i.p., dissolved in cold 0.025 mol/L sodium citrate-buffered solution at pH 4.5, freshly made right before injection) for 5 consecutive days to induce diabetes. Fasting plasma glucose (FPG) levels were determined 2 days after STZ treatment. Diabetic mice were then divided into STZ+vehicle and STZ+ranolazine group (10 mice/group) based on body weight (BW) and blood glucose levels. Age and gender matched non-diabetic mice were used as “normal” controls (n=3). For the following 8 weeks, mice were given either vehicle or ranolazine (20 mg/kg in water, p.o., twice daily). BW and FPG levels were monitored once a week. HbA1c levels were measured using a DCA 2000+ clinical analyzer (Siemens) at 0, 4 and 8 weeks of treatment. At the end of the treatment, pancreases from all groups were collected, fixed in 10% formalin overnight and then embedded in paraffin. HE staining and fluorescent staining were performed to review the islet morphology in all groups.

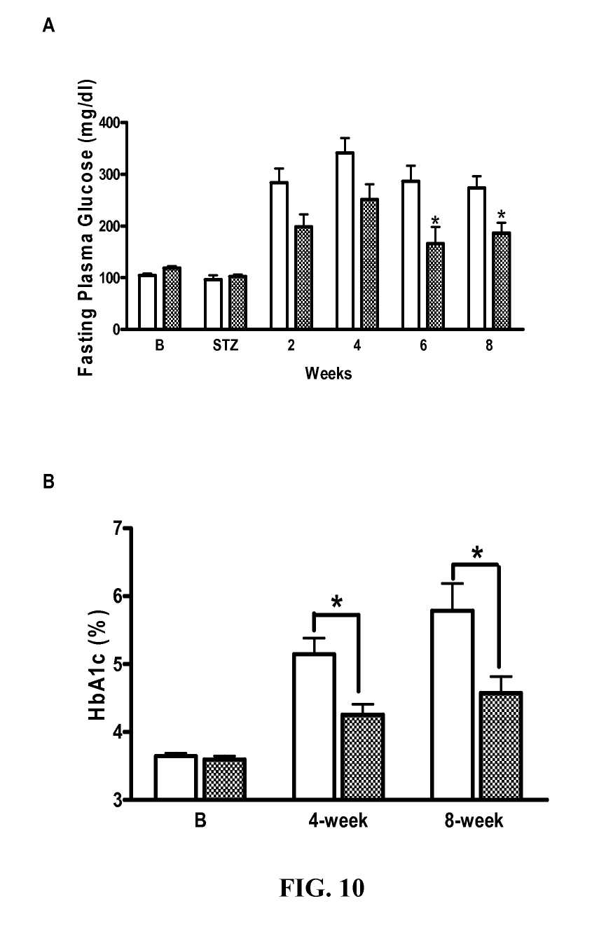

Chronic treatment with ranolazine lowered FPG and HbA1c levels in diabetic mice (FIG. 10). FPG increased significantly with time in both groups after STZ injection (STZ+vehicle group: baseline 108±3 mg/dl to 342±29 mg/dl at week 4, STZ+ranolazine group: baseline 115±2 mg/dl to 264±30 mg/dl at week 4), demonstrating that mice in both groups developed diabetes. However, from week 6 to week 8, FPG was significantly lower in mice treated with ranolazine than that of the mice in the vehicle group (STZ+vehicle: 273±23 mg/dl vs. STZ+ranolazine: 188±20 mg/dl, p<0.05) (FIG. 10A), suggesting that ranolazine slows the progression of diabetes. HbA1c levels also increased significantly in STZ-induced diabetic mice at week 4 and 8 compared to baseline, but HbA1c levels in STZ+ranolazine group were significantly lower than those in STZ+vehicle group after 4-week and 8-week treatment (at week 8 STZ+vehicle: 5.8±0.4% vs STZ+ranolazine: 4.6±0.2%, p<0.05) (FIG. 10B), consistent with the observation in FPG.

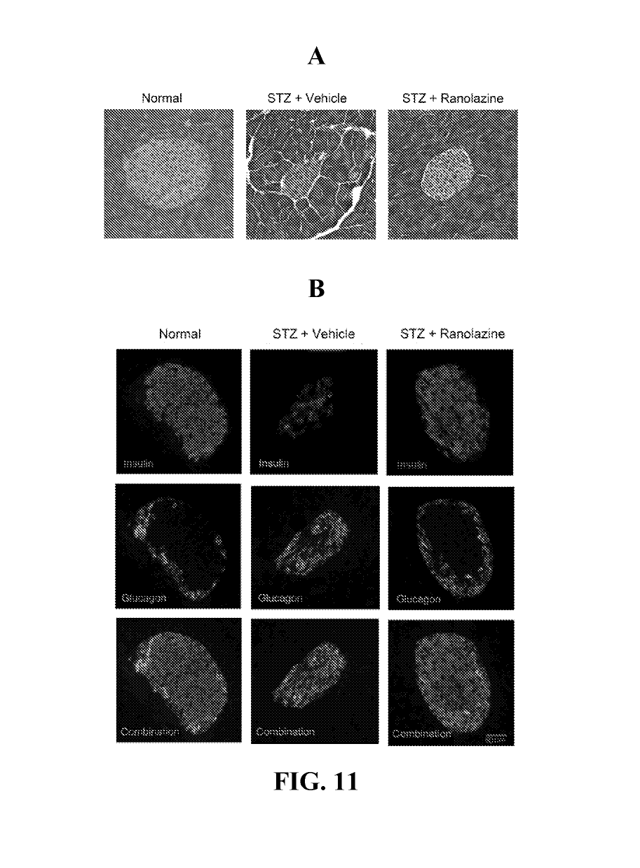

STZ treatment severely decreased β-cell mass and disrupted the islet architecture as compared to the islets from normal mice (FIG. 11A). The clear round islet boundary was destroyed and islet shrinkage was observed in STZ+vehicle group as compared to healthy islets of normal mice. Treatment with ranolazine partially prevented the shrinkage of the islets (FIG. 11A). This result was further confirmed by fluorescence staining of insulin-expressing β-cells and glucagon-expressing α-cells (FIG. 11B). The percentages of total islet (insulin and glucagon) area in STZ+vehicle and STZ+ranolazine were 0.21±0.02%, 0.30±0.03%, respectively (p<0.01). In STZ+vehicle group, insulin-positive area (red staining) was significantly decreased (50±4.6% per islet) whereas glucagon-positive area (green staining) was increased (50±2.1% per islet) as compared to normal group. Ranolazine treatment significantly increased insulin-positive area (69±2.4%, p<0.05) compared with STZ+vehicle group, suggesting partial preservation of functional β-cell mass in pancreas.

### 3.2. Anti-Diabetic Effects of Sodium Channel Blockers Ranolazine and Compound a in Zdf Diabetic Rats, an Animal Model of Type 2 Diabetes

Male ZDF Leprfa/Crl rats were received at 5 weeks of age from Charles River Laboratories Inc., (Wilmington, Mass.) and were acclimated until study initiation at 6 weeks of age. Drugs were given to the animals in Purina 5008 for 10 weeks at doses approximately 170 mg/kg/d of ranolazine, 0.6 mg/kg/d of compound A and 30 mg/kg/d of sitagliptin as positive control. Fasting (12-14 h fast) and non-fasting blood samples were obtained by tail-nick and blood glucose was measured using a Freestyle Lite glucose meter (Abbott Laboratories Inc., Abbott Park, Ill.). HbA1c levels were monitored every other week. Twenty four hour water consumption as surrogate marker for diabetes for each rat was measured at least once per week.

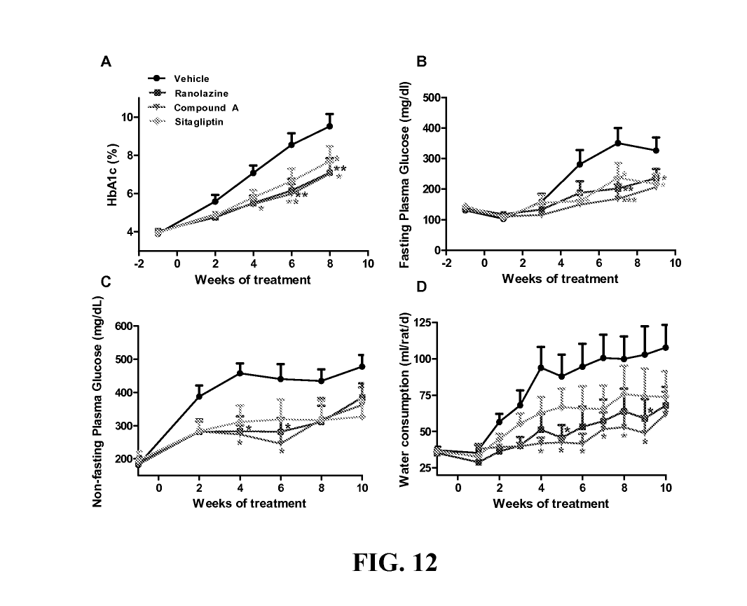

FIG. 12 shows that treatment with ranolazine, compound A or sitagliptin (as a positive control) in ZDF diabetic rats improves HbA1c, fasting and non-fasting glucose, water consumption. At baseline (6 weeks old), HbA1c was 3.9-4.0% in the four groups and in the vehicle group increased to 9.5% by 9 weeks. HbA1c levels were significantly lower in the ranolazine, compound A, sitagliptin treated groups than in the vehicle group at weeks 4, 6 and 8 (FIG. 12A). Fasting glucose levels in vehicle treated animals began to increase by week 5 (11 weeks old) and reached a plateau at week 6 (12 weeks old). Ranolazine, compound A and sitagliptin groups prevented fasting hyperglycemia and fasting plasma glucose levels were significantly lower than vehicle at weeks 7 and 9 (FIG. 12B). Non-fasting glucose was also lower in treatment groups compared to vehicle group (FIG. 12C), consistent with the results for fasting glucose. Water consumption (a surrogate marker for diabetes) in vehicle-treated animals increased from 35 mL/d at 2 weeks (8 weeks old) to approximately 93 mL/d beyond 4 weeks (10 weeks old). In comparison, ranolazine, compound A and sitagliptin treatment significantly prevented the increase in water consumption during diabetes development (FIG. 12D).

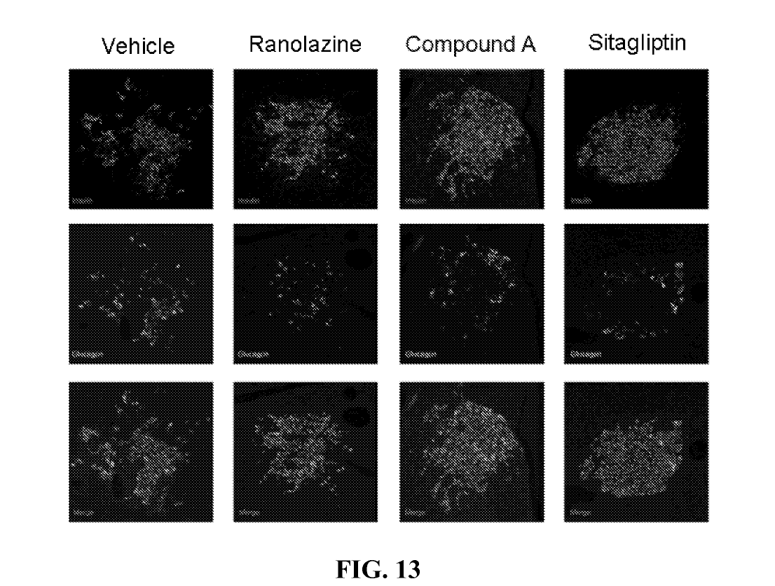

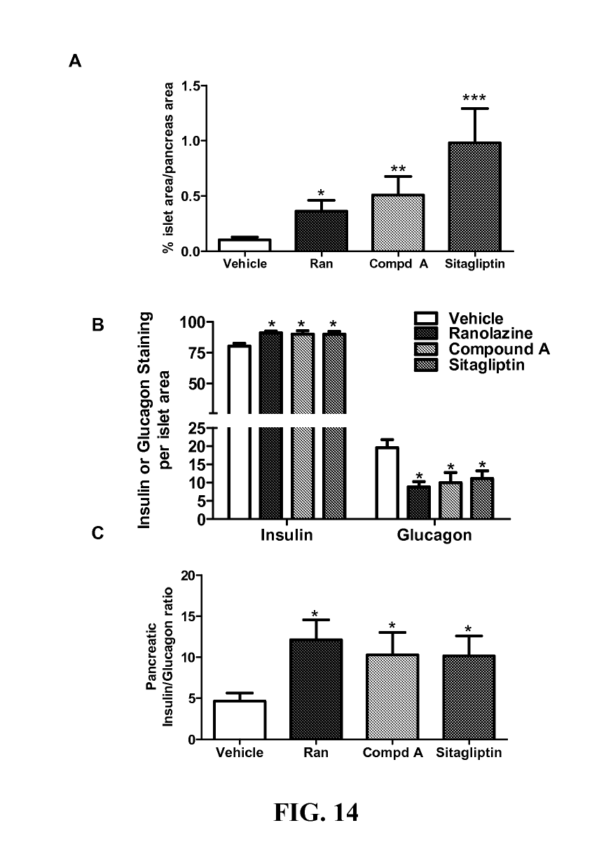

Representative islets from all groups were stained with insulin and glucagon antibodies (FIG. 13). There was significantly more islet area/pancreas area in sections from ranolazine (0.36±0.1%), compound A (0.51±0.16%) and sitagliptin (0.98±0.3%) treated groups compared to vehicle-treated animals (0.1±0.02%)(FIG. 14A), perhaps indicating islet preservation. Consistent with healthier islets, there was significantly more insulin staining per islet with ranolazine (91.2±1.5%), compound A (90.0±2.8%) and sitagliptin (90.0±2.1%) and significantly less staining of glucagon (FIG. 14B). Together these results demonstrate that insulin/glucagon ratios were much higher in ranolazine (12.1±2.4), compound A (10.3±2.7%) and sitagliptin (10.2±2.4%) treated animals compared with vehicle treatment (4.6±1.0) (FIG. 14C) and islets in ranolazine, compound A and sitagliptin groups have higher insulin capacity per islet.

### 4. Sodium Channel Subtypes in Rat and Human Pancreatic Islets

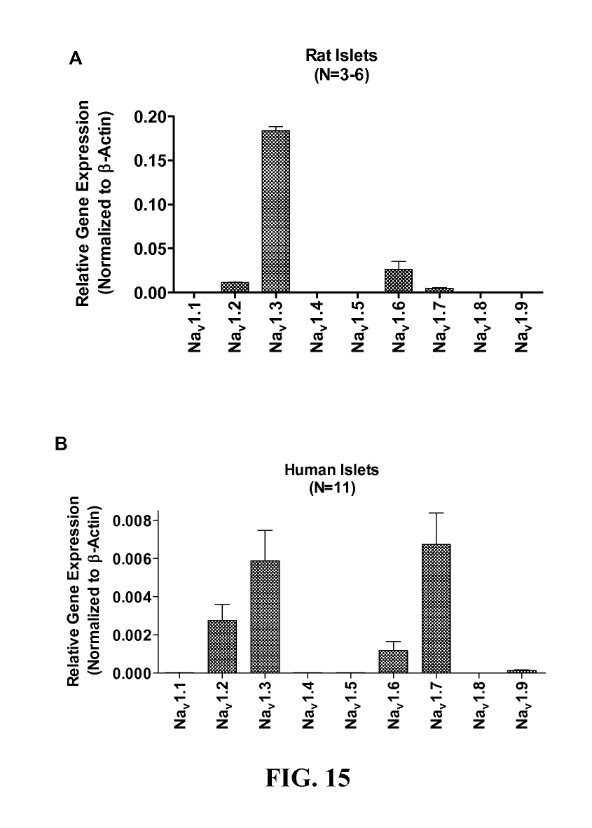

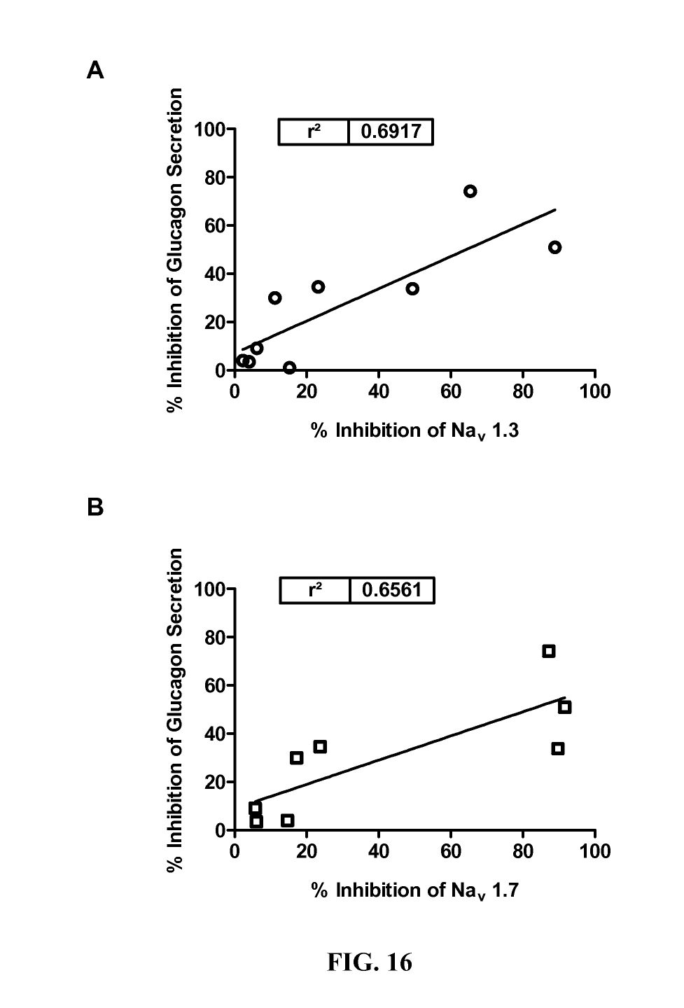

Gene expression of sodium channel subtypes in isolated pancreatic islets from male Sprague Dawley rats and adult human donors was determined using RT-PCR. Na_{v1.3} was found to be the predominant subtype expressed in rat pancreatic islets whereas in human pancreatic islets, Na_{v1.2}, Na_{v1.3} and Na_{v1.7} are highly expressed (FIG. 15). Table 3 shows the blockade of Na_{V1.3} and Na_{V1.7} and inhibition of veratridine-induced glucagon secretion in α-TC1 clone 9 cells by various Na channel blockers at a given concentration. A correlation between inhibition of Na_{v1.3} and Na_{v1.7} channels and glucagon secretion was observed (FIG. 16). Based on the current data it seems that targeting either Na_{v1.3} or Na_{v1.7} may be sufficient to inhibit glucagon secretion for treatment of diabetes.

TABLE 3
Inhibition of Naᵥ 1.3 and Naᵥ 1.7 Na channel isoforms and glucagon secretion
by various Na channel blockers.

| | | hNaᵥ1.3 | hNaᵥ1.7 | Glucagon |
|---|---|---|---|---|
| Cmpd | | VDB | VDB | Secretion |
| No. | Name | % Inhibition | % Inhibition | % Inhibition |
| A | 6-(4-(trifluoromethoxy)phenyl)-3- | 65.42 | 87.17 | 74.17 |
| | (trifluoromethyl)-[1,2,4]triazolo[4,3- | | | |
| | α]pyridine | | | |
| B | 6-(2-methyl-4- | 88.92 | 91.59 | 50.96 |
| | (trifluoromethoxy)phenyl)-3- | | | |
| | (trifluoromethyl)-[1,2,4]triazolo[4,3- | | | |
| | a]pyridine | | | |
| C | N-(3-chloro-4-(trifluoromethyl)benzyl)- | 92.16 | 7.81 | 41 |
| | 4-(N-(5-chlorothiazol-2- | | | |
| | yl)sulfamoyl)benzamide (Pfizer) | | | |
| D | 3-phenoxy-6-(4- | 49.35 | 89.74 | 33.75 |
| | (trifluoromethoxy)phenyl)- | | | |
| | [1,2,4]triazolo[4,3-a]pyridine | | | |
| E | tert-butyl (R)-1-oxo-3-phenyl-1-((R)-1- | 23.18 | 23.73 | 34.53 |
| | (2,2,2-trifluoroethyl)-2,3,4,5-tetrahydro- | | | |
| | 1H-benzo[b]azepin-3-ylamino)propan- | | | |
| | 2-ylcarbamate | | | |
| F | 6-(4-(4-chlorophenoxy)phenyl)-3-(1,1- | 2.32 | 14.72 | 4 |
| | difluoro-2-methoxyethyl)- | | | |
| | [1,2,4]triazolo[4,3-a]pyridine | | | |
| G | N-(2,4′-dichloro-3′- | 4.01 | 6.04 | 3.5 |
| | (trifluoromethyl)biphenyl-4- | | | |
| | yl)methanesulfonamide | | | |
| | Ranolazine | 11.21 | 17.23 | 29.99 |

It will be appreciated that those skilled in the art will be able to devise various arrangements which, although not explicitly described or shown herein, embody the principles of the disclosure and are included within its spirit and scope. Furthermore, all conditional language recited herein is principally intended to aid the reader in understanding the principles of the disclosure and the concepts contributed by the inventors to furthering the art, and are to be construed as being without limitation to such specifically recited conditions. Moreover, all statements herein reciting principles, aspects, and embodiments of the disclosure are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of present disclosure is embodied by the appended claims.

1. A method of reducing the secretion of glucagon from a pancreatic alpha cell, comprising contacting the alpha cell with an agent that suppresses the conduction of sodium ions through sodium channels.

2. The method of claim 1, wherein the alpha secrets a higher level of glucagon as compared to a normal pancreatic alpha cell.

3. A method of lowering the plasma level of HbA1c or glucose, delaying onset of diabetic complications, or treating diabetes in a patient, comprising administering to the patient an effective amount of an agent that suppresses the conduction of sodium ions through sodium channels, wherein the agent is selected from the group consisting of lidocaine, mexiletine, flecamide, amiloride, triamterene, benzamil, A-803467, quinidine, procainamide, disopyramide, tocamide, phenyloin, encamide, moricizine, and propafenone, a local anesthetic, a class I antiarrhythmic agent, an anticonsulsant, and combinations thereof.

4. The method of claim 3, wherein the patient has enhanced glucagon secretion as compared to a normal patient.

5. The method of any one of claims 1-4, wherein the agent is not a compound of Formula I, 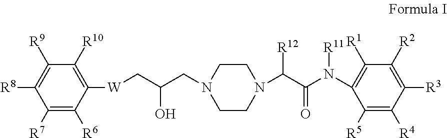
wherein:
R¹, R², R³, R⁴ and R⁵ are each independently hydrogen, lower alkyl, lower alkoxy, cyano, trifluoromethyl, halo, lower alkylthio, lower alkyl sulfinyl, lower alkyl sulfonyl, or N-optionally substituted alkylamido, provided that when R¹ is methyl, R⁴ is not methyl;
or R² and R³ together form —OCH₂O—;
R⁶, R⁷, R⁸, R⁹ and R¹⁰ are each independently hydrogen, lower acyl, aminocarbonylmethyl, cyano, lower alkyl, lower alkoxy, trifluoromethyl, halo, lower alkylthio, lower alkyl sulfinyl, lower alkyl sulfonyl, or di-lower alkyl amino; or
R⁶ and R⁷ together form —CH═CH—CH═CH—; or
R⁷ and R⁸ together form —O—CH₂O—;
R¹¹ and R¹² are each independently hydrogen or lower alkyl; and
W is oxygen or sulfur;
or a pharmaceutically acceptable salt or ester thereof, or an isomer thereof.

6. The method of any one of claims 3-5, wherein the agent is administered intravenously.

7. The method of any one of claims 3-5, wherein the agent is administered orally.

8. The method of any of claims 3-7, wherein the agent is administered in a sustained release formulation.

9. A method of treating diabetes in a human patient, comprising administering to the subject (a) a synergistically therapeutically effective amount of insulin or a drug that increases the production of insulin or sensitivity to insulin and (b) a synergistically therapeutically effective amount of an agent that suppresses the conduction of sodium ions through sodium channels.

10. The method of claim 9, wherein the drug is selected from the group consisting of chlorpropamide, tolbutamide, glyburide, glipizide, glimepiride, reparglinide, nateglinide, pioglitazone and combinations thereof.

11. The method of claim 9 or 10, wherein the agent is selected from the group consisting of lidocaine, mexiletine, flecamide, amiloride, triamterene, benzamil, A-803467, quinidine, procainamide, disopyramide, tocamide, phenyloin, encamide, moricizine, and propafenone, a local anesthetic, a class I antiarrhythmic agent, an anticonsulsant, and combinations thereof.

12. An agent that suppresses the conduction of sodium ions through sodium channels selected from the group consisting of lidocaine, mexiletine, flecamide, amiloride, triamterene, benzamil, A-803467, quinidine, procainamide, disopyramide, tocamide, phenyloin, encamide, moricizine, and propafenone, a local anesthetic, a class I antiarrhythmic agent, an anticonsulsant, and combinations thereof for use in lowering the plasma level of HbA1c or glucose, delaying onset of diabetic complications, or treating diabetes in a patient.

13. A combination of (a) a synergistically therapeutically effective amount of insulin or a drug that increases the production of insulin or sensitivity to insulin and (b) a synergistically therapeutically effective amount of an agent that suppresses the conduction of sodium ions through sodium channels for use in treating diabetes.

14. A method for the manufacture of a medicament for use in lowering the plasma level of HbA1c or glucose, delaying onset of diabetic complications, or treating diabetes in a patient, comprising administering to the patient an effective amount of an agent that suppresses the conduction of sodium ions through sodium channels.

15. A method for the manufacture of a medicament for use in lowering the plasma level of HbA1c or glucose, delaying onset of diabetic complications, or treating diabetes in a patient, comprising administering to the patient an effective amount of an agent that suppresses the conduction of sodium ions through sodium channels wherein the agent is selected from the group consisting of lidocaine, mexiletine, flecamide, amiloride, triamterene, benzamil, A-803467, quinidine, procainamide, disopyramide, tocamide, phenyloin, encamide, moricizine, and propafenone, a local anesthetic, a class I antiarrhythmic agent, an anticonvulsant, and combinations thereof.


SEQUENCE LISTING (40 sequences)

SEQ ID NO: 1 - DNA, 22 bp, Artificial Sequence
  source: /note=“Description of Artificial Sequence Synthetic primer”
agtcaattacatcaggacattt

SEQ ID NO: 2 - DNA, 19 bp, Artificial Sequence
  source: /note=“Description of Artificial Sequence Synthetic primer”
gcagttcacgaatacagtt

SEQ ID NO: 3 - DNA, 19 bp, Artificial Sequence
  source: /note=“Description of Artificial Sequence Synthetic primer”
tggcactagaactgtatca

SEQ ID NO: 4 - DNA, 24 bp, Artificial Sequence
  source: /note=“Description of Artificial Sequence Synthetic primer”
tgtaactggtaatataacttcact

SEQ ID NO: 5 - DNA, 18 bp, Artificial Sequence
  source: /note=“Description of Artificial Sequence Synthetic primer”
aaccctgtctctcaaatg

SEQ ID NO: 6 - DNA, 18 bp, Artificial Sequence
  source: /note=“Description of Artificial Sequence Synthetic primer”
ggcacataactgttcaga

SEQ ID NO: 7 - DNA, 18 bp, Artificial Sequence
  source: /note=“Description of Artificial Sequence Synthetic primer”
tactcagggcattctgtt

SEQ ID NO: 8 - DNA, 18 bp, Artificial Sequence
  source: /note=“Description of Artificial Sequence Synthetic primer”
acactcaagcacacatac

SEQ ID NO: 9 - DNA, 23 bp, Artificial Sequence
  source: /note=“Description of Artificial Sequence Synthetic primer”
ctcctgtatcctgtatcaatcta

SEQ ID NO: 10 - DNA, 20 bp, Artificial Sequence
  source: /note=“Description of Artificial Sequence Synthetic primer”
ttggcttttgtcatttcctt

SEQ ID NO: 11 - DNA, 19 bp, Artificial Sequence
  source: /note=“Description of Artificial Sequence Synthetic primer”
acaaccaactaattgacta

SEQ ID NO: 12 - DNA, 18 bp, Artificial Sequence
  source: /note=“Description of Artificial Sequence Synthetic primer”
ggctgtatgttagagatg

SEQ ID NO: 13 - DNA, 24 bp, Artificial Sequence
  source: /note=“Description of Artificial Sequence Synthetic primer”
catcttaggttcattcatcttagg

SEQ ID NO: 14 - DNA, 21 bp, Artificial Sequence
  source: /note=“Description of Artificial Sequence Synthetic primer”
ggcttggtaggtatgtgataa

SEQ ID NO: 15 - DNA, 18 bp, Artificial Sequence
  source: /note=“Description of Artificial Sequence Synthetic primer”
aatctgaaactgcttctg

SEQ ID NO: 16 - DNA, 18 bp, Artificial Sequence
  source: /note=“Description of Artificial Sequence Synthetic primer”
gtcctcatgttgactcta

SEQ ID NO: 17 - DNA, 21 bp, Artificial Sequence
  source: /note=“Description of Artificial Sequence Synthetic primer”
ttctgaggatctgtggcttgt

SEQ ID NO: 18 - DNA, 23 bp, Artificial Sequence
  source: /note=“Description of Artificial Sequence Synthetic primer”
tctggaaagattactgggtagca

SEQ ID NO: 19 - DNA, 20 bp, Artificial Sequence
  source: /note=“Description of Artificial Sequence Synthetic primer”
tgtacgccaacacagtgctg

SEQ ID NO: 20 - DNA, 21 bp, Artificial Sequence
  source: /note=“Description of Artificial Sequence Synthetic primer”
ccgatccacacggagtacttg

SEQ ID NO: 21 - DNA, 18 bp, Artificial Sequence
  source: /note=“Description of Artificial Sequence Synthetic primer”
gtgcgtgtgtttgtgtac

SEQ ID NO: 22 - DNA, 20 bp, Artificial Sequence
  source: /note=“Description of Artificial Sequence Synthetic primer”
gcagtaaggaacaacatctc

SEQ ID NO: 23 - DNA, 22 bp, Artificial Sequence
  source: /note=“Description of Artificial Sequence Synthetic primer”
tttatttcagcactttcttacg

SEQ ID NO: 24 - DNA, 20 bp, Artificial Sequence
  source: /note=“Description of Artificial Sequence Synthetic primer”
ttcctgtttgggtctcttag

SEQ ID NO: 25 - DNA, 19 bp, Artificial Sequence
  source: /note=“Description of Artificial Sequence Synthetic primer”
accgtccattctaaccatc

SEQ ID NO: 26 - DNA, 19 bp, Artificial Sequence
  source: /note=“Description of Artificial Sequence Synthetic primer”
cgatagcagcaagagattc

SEQ ID NO: 27 - DNA, 18 bp, Artificial Sequence
  source: /note=“Description of Artificial Sequence Synthetic primer”
cgctcttctctgcttctg

SEQ ID NO: 28 - DNA, 20 bp, Artificial Sequence
  source: /note=“Description of Artificial Sequence Synthetic primer”
caatagattgtgccaccttc

SEQ ID NO: 29 - DNA, 21 bp, Artificial Sequence
  source: /note=“Description of Artificial Sequence Synthetic primer”
caccttcactgccatctacac

SEQ ID NO: 30 - DNA, 20 bp, Artificial Sequence
  source: /note=“Description of Artificial Sequence Synthetic primer”
tgcgtaaggctgagacattg

SEQ ID NO: 31 - DNA, 13 bp, Artificial Sequence
  source: /note=“Description of Artificial Sequence Synthetic primer”
tgatgatcctgac

SEQ ID NO: 32 - DNA, 20 bp, Artificial Sequence
  source: /note=“Description of Artificial Sequence Synthetic primer”
gctctcgttgaagtttatgg

SEQ ID NO: 33 - DNA, 23 bp, Artificial Sequence
  source: /note=“Description of Artificial Sequence Synthetic primer”
ctgctgagagtgaagaagaattg

SEQ ID NO: 34 - DNA, 20 bp, Artificial Sequence
  source: /note=“Description of Artificial Sequence Synthetic primer”
gctcgtgtagccataatccg

SEQ ID NO: 35 - DNA, 18 bp, Artificial Sequence
  source: /note=“Description of Artificial Sequence Synthetic primer”
gcatcaggaacggaacag

SEQ ID NO: 36 - DNA, 18 bp, Artificial Sequence
  source: /note=“Description of Artificial Sequence Synthetic primer”
agtgaccagcatcagacc

SEQ ID NO: 37 - DNA, 19 bp, Artificial Sequence
  source: /note=“Description of Artificial Sequence Synthetic primer”
cttcacttccgactctctg

SEQ ID NO: 38 - DNA, 20 bp, Artificial Sequence
  source: /note=“Description of Artificial Sequence Synthetic primer”
gcttaggtaacttcctggag

SEQ ID NO: 39 - DNA, 19 bp, Artificial Sequence
  source: /note=“Description of Artificial Sequence Synthetic primer”
ttcaacaccccagccatgt

SEQ ID NO: 40 - DNA, 23 bp, Artificial Sequence
  source: /note=“Description of Artificial Sequence Synthetic primer”
agtggtacgaccagaggcataca